(19) United States
(12) Patent Application Publication
(10) Pub. No.: US 2004/0038273 A1
(43) Pub. Date: Feb. 26, 2004

(54) Bifunctional tRNA for in vitro selection

(75) Inventors: Charles E. Merryman, Sykesville, MD (US); David P. Bartel, Brookline, MA (US)

Correspondence Address: ROPES & GRAY LLP, ONE INTERNATIONAL PLACE, BOSTON, MA, 02110-2624, US

(73) Assignee: Whitehead Institute for Biomedical Research, Cambridge, MA (US)

(21) Appl. No.: 10/464,802

(22) Filed: Jun. 17, 2003

Related U.S. Application Data
(60) Provisional application No. 60/389,744, filed on Jun. 17, 2002.

(51) Int. Cl. C12Q 1/68; C07H 21/02

(52) U.S. Cl. 435/6; 536/23.1

(57) ABSTRACT

Disclosed are tRNA analogues which comprise a tRNA, such as tRNA^{phe}; a nonstandard amino acid moiety which acts as an acceptor substrate, but not as a donor substrate, for ribosome-directed nonstandard polymer transfer and, thus, is stably linked to the acceptor stem of the tRNA; and a reactive or activatible moiety near or within the anticodon stem loop of the tRNA that can medidate the covalent coupling of the tRNA analogue to mRNA. Also disclosed are nonstandard polymer-tRNA analogue-mRNA fusions; libraries of encoded nonstandard polymers; methods of producing and screening the libraries; and target members and their uses.

### RELATED APPLICATION

This application claims the benefit of the filing date of U.S. provisional application No. 60/389,744, entitled “Bifunctional tRNA for in vitro Selection” by Charles E. Merryman and David P. Bartel, filed Jun. 17, 2002.

This application is related to U.S. provisional application No. 60/082,252, entitled “Method for producing Diverse Libraries of Encoded Peptides,” by Charles E. Merryman and David P. Bartel, filed Apr. 17, 1998 and to U.S. application (application Ser. No. 09/291,704) entitled “Method for producing Diverse Libraries of Encoded Peptides,” by Charles E. Merryman and David P. Bartel, filed Apr. 14, 1999 now U.S. Pat. No. 6,440,695 and a continued prosecution application of “Method for producing Diverse Libraries of Encoded Peptides,” by Charles E. Merryman and David P. Bartel, filed Apr. 2, 2001 and published under publication number US-2002-0031762-A1. The entire teachings of the referenced applications are incorporated herein by reference.

### FUNDING

Work described herein was funded, in whole or in part, by National Institutes of Health, Grant NIH 5 F32 DK10018. The United States government has certain rights in the invention.

### BACKGROUND OF THE INVENTION

Large combinatorial libraries of biopolymers are starting points for isolating new enzymes, binding motifs and other useful molecules. For example, current technologies can generate populations of nucleic acids with complexities on the order of 10¹⁵ molecules and then isolate and identify a single molecule with a desired activity. Random polypeptide populations have greater chemical diversity than do polynucleotides, making them an attractive alternative to nucleic acids. Current systems are limited in their ability to easily generate large complex libraries of polypeptides that are in a form that allows the isolation and identification of rare molecules with a desired activity.

### SUMMARY OF THE INVENTION

The present invention relates to tRNA analogues; polymer-tRNA analogue-mRNA fusions, such as polypeptide-tRNA analogue-mRNA fusions; diverse libraries of encoded polymers, such as encoded polypeptides; and a method of producing the diverse libraries. tRNA analogues of the present invention comprise a tRNA (such as a yeast tRNA); an amino acid moiety that acts as an acceptor substrate, but not as a donor substrate, for ribosome-directed peptidyl transfer; and a reactive or activatible moiety near or within the anticodon stemp loop of the tRNA that can mediate the stable coupling of the tRNA analogue to mRNA. An amino acid moiety is stably linked to the tRNA if the linkage between the two or the chemical environment allows the amino acid moiety to act as an acceptor substrate but not as a donor substrate for ribosome-directed peptidyl transfer. In a specific embodiment, the tRNA analogue is a 3′-amino-3′-deoxyadenosine-substituted tRNA in which the 3′-terminal A of yeast tRNAP^{phe} is replaced or substituted by 3′-amino-3′-deoxyadenosine and then the substituted tRNA is charged with phenylalaine. This tRNA analogue is termed PHE-N-tRNA. In another specific embodiment, the tRNA analogue is a puromycin-substituted tRNA in which the 3′-terminal A of yeast tRNA^{phe} is replaced or substituted by puromycin, with the result that the amino acid moiety is the methoxytyrosine moiety of puromycin. In both embodiments, the reactive or activatible moiety near or within the anticodon stem-loop is the modified Y base of yeast tRNA^{phe}.

Also the subject of this invention are polypeptide-tRNA analogue-mRNA fusions, in which: the polypeptide can be a peptide or polypeptide of any size; the tRNA analogue is as described herein; and the mRNA is mRNA which encodes the polypeptide component of the fusion. In a fusion, the tRNA analogue component is: (a) located between the polypeptide and the mRNA which encodes the polypeptide; (b) linked to the polypeptide by a convalent bond between the terminal amino acid residue of the polypeptide and the amino acid moiety of the tRNA analogue; and (c) linked to the mRNA by crosslinks between a reactive or activatible moiety of the tRNA analogue and the mRNA. In one embodiment, the polypeptide-tRNA analogue-mRNA fusion includes a PHE-N-tRNA (and, thus, the amino acid moiety which acts as an acceptor substrate but cannot act as a donor substrate is phenylalanine) and the tRNA is yeast tRNA^{phe}. In another embodiment, the polypeptide-tRNA analogue-mRNA fusion includes a puromycin-substituted tRNA (and, thus, the amino acid moiety which acts as an acceptor substrate but cannot act as a donor substrate is the methoxytyrosine moiety of puromycin) and the tRNA is yeast tRNA^{phe}.

Diverse libraries or collections of encoded polypeptides are also the subject of this invention. A diverse library or collection comprises the encoded polypeptides, which are the polypeptide-tRNA analogue-mRNA fusions described herein. Methods of producing such libraries are also the subject of this invention.

The invention further relates to a method of screening a diverse encoded polypeptide library to identify target members (library members with desired biological or biochemical properties or activities, such as binding to a particular ligand or enzymatic activity). In one embodiment of screening a diverse encoded polypeptide library to identify a target member or members, the diverse library is initially enriched in molecules with desired properties. This is done, for example, to identify a binding partner or ligand of interest using known enrichment methods, such as affinity enrichment using an immobilized ligand or binding partner or to identify a library member with enzymatic activity by assessing affinity of a library member to a product of a reaction in which the enzyme has modified itself or a substrate to which the library member is attached. Library members identified in this way are target members. In a further step in the method, a library which has been enriched in target members (an enriched fusion or encoded diverse polypeptide library) is amplified and subjected to additional enrichment. For example, the enriched library is reverse transcribed, thereby producing cDNAs of the mRNA components; the cDNAs are, optionally, amplified. The initially produced cDNAs or the resulting PCR products are subjected to in vitro transcription, thereby producing an amplified pool of mRNAs that encode members of the enriched fusion library. The amplified pool of mRNAs is subjected to in vitro translation in the presence of the tRNA analogue of the present invention, producing an amplified version of the enriched encoded polypeptide library. Library members (fusions) amplified in this manner are, optionally, subjected to further enrichment and amplification, as necessary, until target members are enriched to a level where they are present in sufficient numbers to be detected. They are detected using any known method, such as binding to a ligand of interest or catalyzing a reaction of interest. mRNAs of target members are cloned and then individual fusions made from cloned mRNAs are screened for the desired properties (e.g., by ligand binding or catalyzing a reaction of interest). Library members identified in this way are target members, which are also a subject of this invention.

Target members are polypeptide-tRNA analogue-mRNA fusions. The translation products of the enriched mRNA (the polypeptide component of a target member) which display properties of interest and modified or engineered derivatives of the translation products which display properties of interest are target polypeptide fragments. These fragments are also a subject of this invention. As used herein, the term target polypeptide fragments includes fragments released or separated from target members in which they occur and fragments produced or synthesized by another method (e.g., chemical synthesis, mRNA translation in the absence of the tRNA analogue or a recombinant DNA method in which DNA encoding a desired target polypeptide fragment is expressed). Target polypeptide fragments include, but are not limited to, protein catalysts, single-chain monoclonal antibodies, binding pair members (ligand or binding partner), receptors or their ligands, and enzymes and their substrates.

A further subject of the present invention is polymer-tRNA analogue-mRNA fusions in which the polymer is a nonstandard polymer. In addition to the 20 canonical amino acids, the ribosome can polymerize a wide variety of nonstandard monomers and it is polymers of such nonstandard monomers that are present in (are a component of) the nonstandard polymer-tRNA analogue-mRNA fusions that are a subject of this invention. Nonstandard monomers are noncanonical monomers that can be used by the ribosome, first as an acceptor and then as a donor substrate during translation. The term “nonstandard polymer” refers to any polymer containing at least one nonstandard monomer. Thus, a nonstandard polymer of the present invention can be a polymer comprising only nonstandard monomers, or can be a polymer comprising a mix of nonstandard monomers and canonical amino acids. A wide variety of nomomers can be polymerized by the ribosome and are thus included within the nonstandard polymer-tRNA analogue mRNA fusions of the present invention. In conjunction with the system described herein, such monomers can be used to generate encoded polymers that can easily undergo in vitro selection and evolution.

### BRIEF DESCRIPTION OF THE DRAWINGS

FIGS. 1a and 1b are schematic representations of two embodiments of tRNA analogues of the present invention, in which the tRNA portion is stably linked by an amide bond (very thick line) to the amino acid moiety of the tRNA analogue and the reactive or activatible moiety is a modified nucleotide (Y base) which can be used to couple the tRNA analogue to mRNA when the base is activated by UV irradiation.

FIG. 2 is a schematic representation of one embodiment of the present invention, in which steps which occur during in vitro translation are presented and the tRNA analogue is a yeast tRNA^{phe} analogue in which the last nucleotide of the acceptor stem has been replaced by 3-amino-3′-deoxyadenosine.

FIG. 3 is a schematic representation of tRNAs bearing nonstandard monomers that are acceptor and donor substrates and can be used by ribosome. Nu denotes a nucleophile, Any denotes virtually any sidechain (for example, D sidechains and disubstitute sidechains can be used). δ+ and e-neg denote a combination of atoms that result in an electrophilic center for nucleophilic attack (i.e. attack by Nu from a separate tRNA). Azido and aminooxy nucleophiles and methyl or ethyl substitutions of the nucleophilic center are also within the scope of the invention.

### DETAILED DESCRIPTION OF THE INVENTION

The present invention relates to a modified tRNA or functional analogue of tRNA (both referred to herein as a tRNA analogue) that comprises 1) a tRNA or tRNA-like molecule; 2) an amino acid moiety, which is an amino acid, a modified amino acid, or other amino-acid like molecule that can act as an acceptor substrate for ribosome directed peptidyl transfer, but cannot act as a donor substrate for ribosome-directed peptidyl transfer; thus, it is stably linked to the acceptor stem of the tRNA or tRNA-like molecule and 3) a reactive or activatible moiety, which can be any addition, deletion, substitution, modification or alteration of the tRNA (such as addition, deletion, substitution, modification or alteration of one or more bases or nucleotides near or within the anticodon stem-loop of the tRNA), that can mediate the covalent coupling of the tRNA analogue to messenger RNA (mRNA). For example, the activatible moiety can be the naturally occurring modification of guanine (the Y base) that is found in the anticodon loop at position 37 of yeast tRNA^{phe}. Such a tRNA analogue is a bifunctional molecule whose amino-acid moiety can accept, but not donate, a polypeptide that is being synthesized (under the direction of the ribosome) and which can specifically link with the mRNA encoding the polypeptide being synthesized. The tRNA analogue can be, for example, a 3′-amino-3′-deoxyadenosine-substituted tRNA or puromycin-substituted tRNA and the amino acid moiety is any amino acid or the methoxytyrosine moiety of a puromycin-substituted tRNA. The tRNA analogue can be, for example, a tRNA in which the 3′ terminal nucleotide is replaced by 3′-amino-3′-deoxyadenosine and then linked to an amino acid moiety or replaced by puromycin and in which the anticodon loop comprises a reactive or activatible moiety that can mediate covalent coupling of the tRNA analogue to the mRNA. The tRNA analogue can be used to produce diverse encoded polypeptide libraries, which are also the subject of this invention.

The tRNA analogues can comprise any tRNA or tRNA-like molecule (e.g., bacterial, yeast, mammalian, in vitro transcribed, synthesized, tRNA, etc.) which: 1) is modified such that the resulting modified tRNA can form a stable link with a polypeptide being expressed and 2) can make a specific link with the mRNA which encodes the polypeptide being expressed (the encoding mRNA). In a specific embodiment, the 3′ end of the tRNA is modified such that 3′-amino-3′-deoxyadenosine replaces the A base at the 3′ end and then the substituted tRNA is charged with phenylalanine. In another specific embodiment, the 3′ end of the tRNA is modified such that puromycin replaces the A base at the 3′ end. The tRNA analogue can contain any modification which can form the desired stable peptide-tRNA link. As a result of the formation of a stable peptide-tRNA link, protein synthesis is stalled, presumably after translocation of the peptide-tRNA analogue complex to the P site. The specific link between the tRNA analogue and the mRNA which encodes the polypeptide being produced can be formed by activation of an activatible group or via a reactive group in the tRNA (e.g., the Y base) or mRNA.

In one embodiment, the tRNA analogue is produced by replacing the 3′ terminal nucleotide (e.g., the 3′ terminal A) of yeast tRNA^{phe} by 3′-amino-3′-deoxyadenosine and charging the tRNA with phenylalanine. In another embodiment, the tRNA analogue is produced by replacing the 3′ terminal nucleotide (e.g., the 3′ terminal A) of yeast tRNA^{phe} by puromycin. Yeast tRNA^{phe} is useful because it naturally contains a modified guanine base (the Y base) at position 37 that serves as an activatible group that becomes reactive when exposed to UV light, coupling the tRNA analogue to an mRNA. After aminoacylation with phenylalanine, the 3′-amino-3′-deoxyadenosine substitution transforms the yeast tRNA^{phe} into a tRNA analogue (a 3′-amino-3′-deoxyadenosine substituted tRNA) that contains phenylalanine linked to the RNA 3′ terminus by an amide bond. In this embodiment, the amino acid moiety is the phenylalanine moiety of the 3′-amino-3′-deoxyadenosine-substituted tRNA. Alternatively, puromycin substitution transforms the yeast tRNA^{phe} into a tRNA analogue (a puromycin-substituted tRNA) that contains methoxytyrosine linked to the RNA 3′ terminus by an amide bond. In this embodiment, the amino acid moiety is the methoxytyrosine moiety of the puromycin-substituted tRNA. In both embodiments, the amide linkage between the RNA and the phenylalanine or methoxytyrosine prevents the peptidyl-tRNA analogue from being a suitable donor substrate for ribosome-directed peptidyl transfer and, thus, when a polypeptide is transferred to the amino-acid moiety of the tRNA analogue, the polypeptide becomes stably connected to the tRNA analogue.

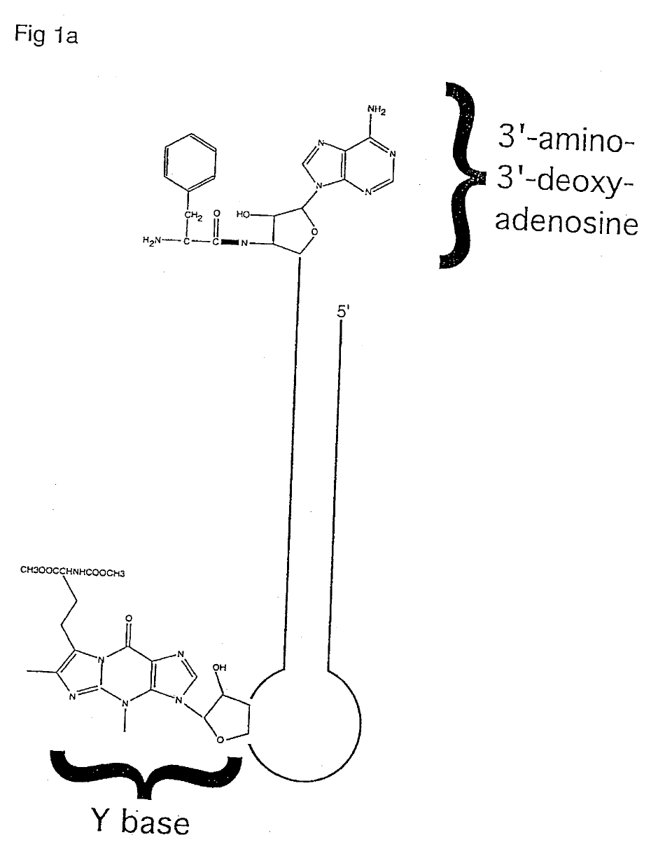

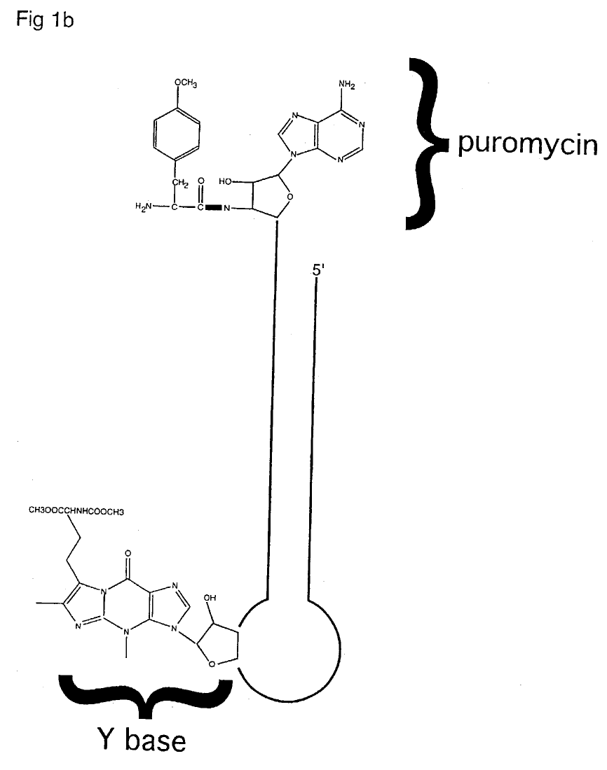

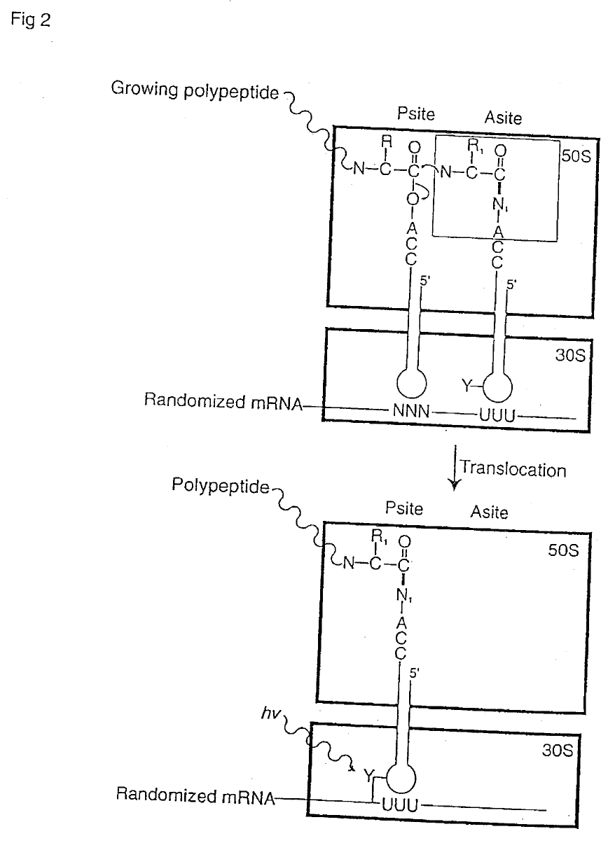

FIGS. 1a and 1b are schematic representations of these embodiments of the present invention. FIGS. 1a and 1b show tRNA analogues that contain an amide bond (very thick line) stably linking the tRNA portion and the amino-acid moiety of the analogue. In FIG. 1a, the tRNA analogue is 3′-amino-3′-deoxyadenosine substituted and the amino acid moiety is a phenylalanine moiety. In FIG. 1b, the tRNA analogue is puromycin substituted and the amino acid moiety is a methoxytyrosine moiety. It also shows the Y base, which is a modified nucleotide which can be used to couple the tRNA analogue to mRNA when the base is activated by UV irradiation. The tRNA portion of the molecule is represented schematically by a thick line. FIG. 2 illustrates the action of the tRNA analogue of this embodiment of the present invention. During translation, when the ribosome reaches a phenylalanine codon (UUU or UUC) in the mRNA, the methoxytyrosine portion (amino-acid moiety) of the tRNA analogue is used as an acceptor substrate and is joined to the carboxy terminus of the nascent polypeptide; because the methoxytyrosine is joined to the tRNA portion of the tRNA analogue by an amide bond, translation stalls. The stalled polypeptide-tRNA^{phe} can be crosslinked to the mRNA (through the Y base of the tRNA^{phe} that was used to make the tRNA analogue) by mild UV irradiation.

The present invention also relates to a diverse library or collection of encoded polypeptides which each comprise three components that are fused into a single molecule that can be produced and manipulated in vitro. Each encoded polypeptide molecule of the library, referred to as a polypeptide-tRNA analogue-mRNA fusion, comprises a polypeptide of any length, an mRNA encoding the polypeptide and a tRNA analogue that is located between the polypeptide and its encoding mRNA and forms a link between these two components. The mRNA component of each fusion can be an mRNA or mRNA-like molecule (of any sequence or length) that is translated by the ribosome. The mRNA component contains elements required for its expression and elements (e.g., primer binding sites) that permit it to be reverse transcribed and then amplified and/or cloned. The tRNA analogue component of each fusion is as described above. That is, the tRNA analogue component, comprises 1) a tRNA or tRNA-like molecule; 2) an amino acid moiety which can act as an acceptor substrate, but not as a donor substrate, for ribosome-directed peptidyl transfer and 3) an activatible moiety that mediates the covalent coupling of the tRNA analogue to messenger RNA.

The diverse library or collection of encoded polypeptides is produced by combining the tRNA analogue with an in vitro translation mixture that contains a diverse mixture of mRNA sequences (such as a randomized pool of mRNAs) and incubating the mixture under conditions appropriate for in vitro translation of mRNA sequences and formation of the linkage between the tRNA analogue and the mRNA. Translation of the mRNAs is stalled as a result of the presence of the stably linked amino acid moiety of the tRNA analogue. At first, members of the diverse collection of mRNAs are translated normally: When a codon is reached, a matching aminoacyl tRNA is selected by the ribosome and bound to the A site. The previously synthesized portion of the nascent polypeptide (which is attached to a P-site bound tRNA) is transferred to the amino acid on the newly selected aminoacyl tRNA (effectively extending the length of the encoded polypeptide by one amino acid) and the tRNA previously bound to the P site is replaced by the newly selected tRNA, which now becomes the P-site bound tRNA. The ribosome uses the now empty A site to match another codon and aminoacyl tRNA. Repetition of this cycle translates mRNA sequences into their corresponding polypeptide sequences. For members of the mRNA of the library this normal translation ends when the tRNA analogue (instead of a normal aminoacyl tRNA) is selected by the ribosome and the ribosome binds the tRNA analogue to the A site. The polypeptide is then transferred to the amino-acid moiety of the tRNA analogue. The stable linkage between the amino-acid moiety and the RNA portion of the tRNA analogue prevents the polypeptide from being further transferred and, thus, translation is stalled with the polypeptide stably attached to the tRNA analogue, which is bound to the mRNA that encodes the polypeptide. Prior to, concurrent with, or after transfer of the polypeptide to the tRNA analogue, the second functionality of the tRNA analogue is used to covalently couple the tRNA analogue to the mRNA. In this way the bifunctional tRNA analogue is used to generate the desired library of polypeptide-tRNA analogue-mRNA fusions.

In one embodiment, the tRNA analogue can be yeast tRNA^{phe}, where the 3′ terminal nucleotide (e.g., the 3′ terminal A of yeast tRNA^{phe}) is replaced by 3′-amino-3′-deoxyadenosine and then charged with phenylalanine. In another embodiment, the tRNA analogue can be yeast tRNA^{phe}, where the 3′ terminal nucleotide (e.g., the 3′ terminal A of yeast tRNA^{phe}) is replaced by puromycin. The resulting tRNA analogues are bifunctional and during in vitro protein synthesis, they can form a stable link (as a result of ribosome directed peptidyl transfer) with the carboxy-terminal amino acid of a polypeptide being produced and a stable link with the mRNA encoding that polypeptide (by the action of mild UV irradiation). FIG. 2 is a schematic representation of one embodiment of the present invention. It represents steps which occur during in vitro translation that link the growing polypeptide to the tRNA analogue (top panel) and that link the tRNA analogue to the mRNA (bottom panel). As illustrated (top panel), a normal polypeptidyl containing tRNA is located in the P site and a tRNA analogue (e.g., PHE-N-tRNA in which 3′-amino-3′-deoxyadenosine is boxed), is located in the A site of a ribosome. Peptidyl transfer (represented by small arrows in the top panel) occurs, resulting in formation of a polypeptide-tRNA fusion by transfer of the peptide being produced to the amino acid moiety of the tRNA analogue (the amide link between the amino-acid moiety and the RNA portion of the tRNA analogue is shown as a very thick line). This amide link stalls protein synthesis, presumably after translocation of the polypeptide-tRNA analogue from the A site to the P site (bottom panel). Exposure of the stalled complexes to UV irradiation results in crosslinking of the polypeptide-tRNA analogue to the mRNA through the Y base of the tRNA analogue, thus generating the desired polypeptide-tRNA analogue-mRNA fusion.

The present invention also relates to the encoded polypeptide (polypeptide-tRNA analogue-mRNA fusions) which comprise the diverse libraries or collections; methods of generating or producing the diverse libraries; a method of identifying and, optionally, amplifying members of the encoded nonstandard polymer library (referred to as target members) which have desired characteristics; target members of the library identified by the method and fragments of the target members (e.g., polypeptide, fragments of polypeptides, or fragments of polypeptide-tRNA analogue-mRNA fusions.)

In the method of generating libraries of encoded polypeptides (diverse collections of polypeptide-tRNA analogue-mRNA fusions), mRNA, tRNA analogues (e.g., PHE-N-tRNA) and an appropriate in vitro translation mixture (e.g., bacterial translation mixtures, such as from E. coli; eucaryotic translation mixtures, such as mixtures from wheat germ and rabbit reticulocytes, or translation mixtures from other organisms) are combined to produce a combination. The resulting combination is maintained under conditions appropriate for translation of the mRNAs, formation of stable peptide-tRNA analogue fusions (linkage between the terminal amino acid residue of the polypeptide being expressed and the stably linked amino-acid moiety, e.g., the phenylalanine of PHE-N-tRNA), and formation of a covalent linkage fusing the tRNA analogue with the mRNA. The in vitro translation mixture is a combination of biological reagents and cellular components which translate mRNAs to produce the polypeptides they encode. If the tRNA portion of the tRNA analogue is yeast tRNA^{phe}, then crosslinking is effected by subjecting the ribosome-bound, stalled polypeptide-tRNA analogue-mRNA complex to UV irradiation.

Once a diverse encoded polypeptide library has been produced, it can be screened, using known methods, to identify target members which are fusions with desired characteristics. A key advantage of the encoded polypeptide library of the present invention, which is comprised of polypeptide-tRNA analogue-mRNA fusions, is that even members which occur in small numbers (rare members) and are of interest because of desired biological or biochemical properties (e.g., binding to a particular ligand, enzymatic activity) can be enriched and then identified by amplification, cloning and sequencing of their respective mRNAs.

A diverse library of encoded polypeptides can be enriched in molecules with the desired properties using known methods, to identify target members. Methods by which target members of the library can be enriched include affinity enrichment using immobilized ligand or binding partner and, for enzymatic activity, affinity to a product of a reaction in which the enzyme has modified itself (with, for example, a mechanism-based inhibitor) or a substrate to which it is attached (e.g., Williams, K. P. and D. P. Bartel, “In Vitro Selection of Catalytic RNA”, pp. 367-381 In: Catalytic RNA, (Fritz Eckstein and David M. J. Lilley, Ed.), Springer, (1996)).

Furthermore, libraries enriched in target members can be amplified and subjected to additional enrichment. For example, a library of fusions that has been enriched for a desired activity (an enriched encoded polypeptide library) can be reverse transcribed, producing the cDNAs of the mRNA components. The cDNAs can then be amplified (e.g., by PCR or other amplification methods). The resulting PCR products are subjected to in vitro transcription, resulting in production of an amplified pool of mRNAs that encode the members of the enriched fusion library. In vitro translation of this pool in the presence of the tRNA analogue links the mRNAs to their translation products, producing an amplified version of the enriched encoded polypeptide library. Fusions amplified in this way are subjected to further enrichment and amplification, which is repeated as necessary until target members are enriched to the desired extent (e.g., enriched to a level where they are present in sufficient numbers to be detected by binding to a ligand of interest or catalyzing a reaction of interest). After sufficient enrichment, mRNAs of target members are cloned and individual fusions can be screened for the desired function. The translation product of the mRNA or a fragment of the translation product can also be screened for activity without attachment to the mRNA.

The method of the present invention of identifying members of a diverse library of encoded polypeptides which exhibit a desired activity is carried out as follows: A diverse library, whose members are polypeptide-tRNA anlogue-mRNA fusions, is produced by combining: 1) mRNAs which encode polypeptides; 2) tRNA analogues, which each comprise: a) a tRNA; b) an amino acid moiety which can act as an acceptor substrate, but not as a donor substrate, for ribosome-directed peptidyl transfer and c) a reactive or activatible moiety near or within the anticodon stem-loop that can mediate (mediates) the covalent coupling of the tRNA analogue to mRNA; and 3) an appropriate in vitro translation mixture, thereby producing a combination. The resulting combination is maintained under conditions appropriate for translation of the mRNAs to produce the encoded polypeptides and formation of a stable amino acid-tRNA analogue bond between the terminal amino acid residue of a polypeptide produced and the amino acid moiety present in the tRNA analogue, to form polypeptide-tRNA analogue fusions, thereby producing a mixture which contains stalled ribosomes that contain polypeptide-tRNA analogue fusions. The mixture which contains stalled ribosomes that contain the fusions is exposed to conditions which favor the crosslinking the tRNA analogue and the mRNA which encodes the polypeptide of the polypeptide-tRNA analogue fusion. As a result, polypeptide-tRNA analogue-mRNA fusions are produced, thereby producing a diverse library of encoded polypeptides. The diverse library of encoded polypeptides is enriched for members which exhibit a desired activity, thereby producing an enriched diverse library comprised of polypeptide-tRNA analogue-mRNA fusions. The resulting enriched diverse library is amplified by: reverse transcribing the mRNA components of the fusions, thereby producing the corresponding cDNA; amplifying and transcribing in vitro the corresponding cDNA, thereby producing a pool of amplified, enriched mRNA from the corresponding cDNA; combining the pool of amplified, enriched mRNA with an appropriate in vitro translation mixture and tRNA analogues (as described above), thereby producing a combination; maintaining the combination under conditions appropriate for translation of the mRNA to produce the encoded polypeptides and formation of a stable amino acid-tRNA analogue bond between the terminal amino acid residue of a polypeptide produced and the amino acid moiety present in the tRNA analogue, to form polypeptide-tRNA analogue fusions, thereby producing an amplified enriched mixture which contains stalled ribosomes that contain polypeptide-tRNA analogue fusion; and exposing the amplified enriched mixture which contains stalled ribosomes that contain polypeptide-tRNA analogue fusions to conditions which favor crosslinking of the tRNA analogue and the mRNA which encodes the polypeptide of the polypeptide-tRNA analogue fusion. The steps of enriching the diverse library and amplifying the enriched diverse library are repeated, as necessary, until members which exhibit the desired activity are present in sufficient number to be detected. Subsequently, members which exhibit the desired activity are detected. As a result, members of the diverse library which exhibit the desired activity are identified.

The translation products of the enriched mRNA (the polypeptide component of the target members), such as polypeptides which display activities of interest (e.g., ligand binding or catalytic activity), as well as engineered derivatives of these translation products which display activities of interest, are referred to as target polypeptide fragments. These target polypeptide fragments are also the subject of this invention. Target polypeptide fragments can be released or separated from target members in which they occur, using known methods (e.g., enzymes which cleave RNA), or they can be synthesized without attachment to the mRNA (e.g., using chemical synthesis or mRNA translation in the absence of the tRNA analogue. They can be used, for example, as diagnostic or therapeutic reagents (e.g., single-chain monoclonal antibodies), protein catalysts, members of binding pairs, receptors or their ligands, enzymes or enzyme substrates. Once a polypeptide fragment which has desired characteristics has been identified, it can be produced using known methods (e.g., production in an appropriate expression system, chemical synthesis).

Ribonucleoprotein fragments of the target members are also the subject of this invention. They can be used, for example, as enzymes or ligands.

A further subject of the present invention is polymer-tRNA analogue-mRNA fusions in which the polymer is a nonstandard polymer. In addition to the 20 canonical amino acids, the ribosome can polymerize a wide variety of nonstandard monomers and it is polymers of such nonstandard monomers that are present in (are a component of) the nonstandard polymer-tRNA analogue-mRNA fusions that are a subject of this invention. Nonstandard monomers are noncanonical monomers that can be used by the ribosome, first as an acceptor and then as a donor substrate during translation. A wide variety of monomers can be polymerized by the ribosome and, thus, included within nonstandard polymer-tRNA analogue-mRNA fusions of the present invention. In conjunction with the system described herein, such monomers can be used to generate encoded polymers that can easily undergo in vitro selection and evolution. There are many types of nonstandard monomers that the ribosome can polymerize. Nonstandard monomers can be subdivided into three major classes: those with nonstandard sidechains, those with nonstandard backbones, and those with nonstandard sidechains and backbones. The third class is a hybrid of the first two and is not discussed below.

The present invention also relates to a diverse library or collection of nonstandard polymers which each comprise three components that are fused into a single molecule that can be produced and manipulated in vitro. Each nonstandard polymer molecule of the library, referred to as a nonstandard polymer-tRNA analogue-mRNA fusion, comprises a nonstandard polymer of any length, an mRNA encoding the nonstandard polymer and a tRNA analogue that is located between the nonstandard polymer and its encoding mRNA and forms a link between these two components. The mRNA component of each fusion can be an mRNA or mRNA-like molecule (of any sequence or length) that is translated by the ribosome. The mRNA component contains elements required for its expression and elements (e.g., primer binding sites) that permit it to be reverse transcribed and then amplified and/or cloned. The tRNA analogue component of each fusion is as described above. That is, the tRNA analogue component, comprises 1) a tRNA or tRNA-like molecule; 2) a amino acid moiety which can act as an acceptor substrate, but not as a donor substrate, for ribosome-directed peptidyl transfer and 3) an activatible moiety that mediates the covalent coupling of the tRNA analogue to messenger RNA.

The diverse library or collection of encoded nonstandard polymers is produced by combining the tRNA analogue with an in vitro translation mixture that contains a diverse mixture of mRNA sequences (such as a randomized pool of mRNAs) and incubating the mixture under conditions appropriate for in vitro translation of mRNA sequences and formation of the linkage between the tRNA analogue and the mRNA. Translation of the mRNAs is stalled as a result of the presence of the stably linked amino acid moiety of the tRNA analogue. At first, members of the diverse collection of mRNAs are translated to incorporate nonstandard monomers. To achieve this, the translation mixture contains at least one mischarged tRNA. The term “mischarged tRNA” refers to a tRNA in which a nonstandard monomer, instead of an amino acid, is attached. The mischarged tRNA can act as both acceptor and donor substrate by the ribosome. For a particular translation mix in which a set of mischarged tRNA is added, there is correspondence between the sequence of the mRNA and the sequence of polymer produced. In this sense, the mRNA encodes a particular sequence of polymer. The exact sequence of the polymer depends on the entity of the monomers engineered to a tRNA. When a codon is reached, a matching aminoacyl tRNA or a mischarged tRNA is selected by the ribosome and bound to the A site. The previously synthesized portion of the nascent polymer (which is attached to a P-site bound tRNA) is transferred to the monomer moiety (it can be an amino acid or a nonstandard monomer) on the newly selected aminoacyl tRNA or a mischarged tRNA (effectively extending the length of the encoded polymer by one monomer) and the tRNA previously bound to the P site is replaced by the newly selected tRNA, which now becomes the P-site bound tRNA. The ribosome uses the now empty A site to match another codon and aminoacyl tRNA or mischarged tRNA. Repetition of this cycle translates mRNA sequences into their corresponding polymer sequences. For members of the mRNA of the library this normal translation ends when the tRNA analogue (instead of a normal aminoacyl tRNA or a mischarged tRNA) is selected by the ribosome and the ribosome binds the tRNA analogue to the A site. The polymer is then transferred to the amino acid moiety of the tRNA analogue. The stable linkage between the amino acid moiety and the RNA portion of the tRNA analogue prevents the polymer from being further transferred and, thus, translation is stalled with the polymer stably attached to the tRNA analogue, which is bound to the mRNA that encodes the polymer. Prior to, concurrent with, or after transfer of the polymer to the tRNA analogue, the second functionality of the tRNA analogue is used to covalently couple the tRNA analogue to the mRNA. In this way the bifunctional tRNA analogue is used to generate the desired library of nonstandard polymer-tRNA analogue-mRNA fusions.

The tRNA analogue can be engineered so that its amino acid moiety is replaced with a nonstandard monomer. The nonstandard monomer in a tRNA analogue is modified such that it acts as an acceptor substrate, but not a donor substrate during translation. All the embodiments for the tRNA analogue with an amino acid applies to the tRNA analogue with such a nonstandard monomer.

The present invention also relates to the encoded nonstandard polymer (nonstandard polymer-tRNA analogue-mRNA fusions) which comprise the diverse libraries or collections; methods of generating or producing the diverse libraries; a method of identifying and, optionally, amplifying members of the encoded nonstandard polymer library (referred to as target members) which have desired characteristics; target members of the library identified by the method and fragments of the target members (e.g., nonstandard polymers, fragments of nonstandard polymers, or fragments of nonstandard polymer-tRNA analogue-mRNA fusions.)

In the method of generating libraries of encoded nonstandard polymers (diverse collections of nonstandard polymer-tRNA analogue-mRNA fusions), mRNA, tRNA analogues (e.g., PHE-N-tRNA) and an appropriate in vitro translation mixture (e.g., bacterial translation mixtures, such as from E. coli; eucaryotic translation mixtures, such as mixtures from wheat germ and rabbit reticulocytes, or translation mixtures from other organisms) are combined to produce a combination. The translation mixture for generating nonstandard polymers may contain EFTu, EFG, fmet-tRNA, mRNA, ribosomes, the bifunctional tRNA and at least one mischarged tRNA in an appropriate composition. The resulting combination is maintained under conditions appropriate for translation of the mRNAs, formation of stable nonstandard polymer-tRNA analogue fusions (linkage between the terminal residue of the nonstandard polymer being expressed and the stably linked amino acid moiety, and formation of a covalent linkage fusing the tRNA analogue with the mRNA. The in vitro translation mixture is a combination of biological reagents and cellular components which translate mRNAs to produce the nonstandard polymers they encode. If the tRNA portion of the tRNA analogue is yeast tRNA^{phe}, then crosslinking is effected by subjecting the ribosome-bound, stalled nonstandard polymer-tRNA analogue-mRNA complex to UV irradiation.

Once a diverse encoded nonstandard polymer library has been produced, it can be screened, enriched, amplified and subjected to additional enrichment using methods described above for a polypeptide library. Furthermore, members of a diverse library of encoded nonstandard polymers which exhibit a desired activity can be identified as described for the library of polypeptides.

The translation products of the enriched mRNA (the nonstandard polymer component of the target members), such as nonstandard polymers which display activities of interest (e.g., ligand binding or catalytic activity), as well as engineered derivatives of these translation products which display activities of interest, are referred to as target nonstandard polymer fragments. These target nonstandard polymer fragments are also the subject of this invention.

1. Nonstandard Sidechains.

Sidechains with elements from Groups IA, VIII, IVB, VB, VIB, and VIIB of the periodic table are used by the ribosome, indicating that the ribosome does not discriminate at the elemental level. In addition to sidechains with standard covalent bonds and double bonds, nonstandard triple bonds and coordination compounds are ribosomal substrates. These compounds show that the ribosome can polymerize sidechains with nonstandard bonds, and thus it does not discriminate at the level of chemical bonding. Furthermore, they show that sidechains containing orbitals with nonstandard hybridization formats are acceptable.

Sidechains with a wide variety of polar, nonpolar, positively charged, negatively charged, and free radical sidechains are polymerized by the ribosome. Reactive, inert, absorbing, excitable and fluorescent sidechains are also found. These compounds show that sidechains with nonstandard chemical and physical properties are ribosomal substrates.

The size of acceptable sidechains varies from a molecular weight of 1 for the canonical amino acid glycine to over 460 for nonstandard sidechains. Thus, the ribosome is quite indiscriminant with respect to sidechain size, volume and related parameters. Furthermore, the sidechains can be linear, branched, cyclic, multicyclic, or heterocyclic. Sidechains with many different stereochemistries and planarities have been identified. These compounds show that acceptable sidechains have very few limits on their structural organization. Collectively, these data represent hundreds of unique sidechains with a wide range of structural, chemical and physical properties. Furthermore, the lack of discrimination by the ribosome at the elemental, bonding, and orgainizational levels suggest that there is an unlimited number of nonstandard sidechains that could be used.

2. Nonstandard Backbones.

The repeating unit of a canonical backbone is 3 units long-(Nitrogen-Carbon-Carbonyl)-. Polymerization is the result of attack by a primary or secondary amine at the carbonyl carbon of an ester bond to form an amide backbone. The stereochemistry at the alpha carbon is always of the L-form except in the case of glycine which is not stereoactive.

Although relatively inefficient, extension of the canonical backbone to 4 units (N-C-C-Car, N-N-C-Car, N-O-C-Car) is possible (Ellman, J. A. et al. (1992). Science 255, 197-200) (Eisenhauer and Hecht Biochemistry 2002 Vol 41 pp. 11472-11478). Furthermore, the present system eliminates some of the translational steps that select against these types of backbones and thus, it should be more efficient. The attacking nitrogen has also been substituted with oxygen, sulfur and azides and aminooxy groups to make ester (Fahnestock, S. and Rich, A. (1971). Science 173, 340-343), thioester (Gooch, J. and Hawtrey, A. O. (1975). Biochem. J. 148, 209-220), and mixed amide backbones (Eisenhauer and Hecht Biochemistry 2002 Vol 41 pp. 11472-11478) and aminooxy backbones (Eisenhauer and Hecht Biochemistry 2002 Vol 41 pp. 11472-11478). The attacking nitrogen has been methylated or ethylated to make peptoid like backbones (Ellman, J. A. et al. (1992). Science 255, 197-200). The ester bond formed with tRNA (which is the leaving group) has been substituted with thiocarbonyl (Victorova, L. S. et al. (1976). FEBS Letters 68, 215-218) and phosphinoesters (Tarusova, N. B. et al. (1981). FEBS Letters 130, 85-87) to form thioamide and phospinoamide backbones. The stereochemistry of the backbone can be D (Ellman, J. A. et al. (1992). Science 255, 197-200), or racemic as in the case of alpha, alpha disubtituted amino acids (Ellman, J. A. et al. (1992). Science 255, 197-200). Furthermore, the ribosome itself can be modified to increase the range of acceptable backbones (See U.S. Pat. No. 6,358,713 and Dedkova et al., J. Am. Chem. Soc. 2003 published on web Mar. 13, 2003). These data show that the ribosome can use a wide variety of backbones, or mixed sets of backbones and that the constraints that exist can be altered to provide an even wider range of possible backbones.

In vitro selection is a powerful approach for generating novel aptamers and catalysts. Currently, several methods are being developed to extend this technique to proteins. In principal, selection methods could be applied to any library whose members can be replicated. Here, Applicants describe a bifunctional tRNA that fuses translation products to their mRNAs. The utility of peptide-tRNA-mRNA fusions for in vitro selection was illustrated by the selective enrichment of tagged peptides—together with their mRNAs—by affinity chromatography. The present system can generate libraries larger than 10¹¹. Because library members can be copied and amplified, they provide a means for applying in vitro selection procedures to peptides and proteins. Furthermore, because the system is amenable to translation with misacylated tRNAs, a wide range of unusual monomers could be used to make libraries of nonstandard polymers for selection experiments.

Exemplification

The present invention is illustrated by the following examples, which are not intended to be limiting in any way.

1. Introduction

In vitro selection is regularly used to search libraries of nucleic acids for rare molecules with desirable functions. Molecules with specific functions are isolated from libraries with more than 10¹⁵ sequences through iterative rounds of selection and amplification; molecules that fulfill the selective criteria increase in representation, and amplification increases their number. Thus, with each round, the functional molecules replace less capable members of the initial population. Although nucleic acids are endowed with recognition and catalytic potential, the application of in vitro selection to polymers with greater chemical diversity would be beneficial. Toward this end, several methods have been developed to attach peptides and proteins to their encoding DNAs or mRNAs [5-9]. Such fusions contain the essential elements needed for selection and amplification: a potentially active protein and a corresponding nucleic acid sequence that stores the information needed to make copies of the protein. In phage display [5] and plasmid display [6], fusion proteins that associate with their encoding nucleic acids are expressed in vivo; the cell membrane encapsulates corresponding protein and nucleic acid sequences during complex formation. Ribosome display [7] relies on the integrity of stalled translation complexes to maintain a link between an mRNA and its protein product. With mRNA display [8, 9], covalent protein-mRNA fusions are formed on stalled translation complexes; the 3′-ends of mRNAs are modified in such a way that they stall protein synthesis, enter the A site of the ribosome, and act like an aminoacyl tRNA to become attached to their protein products.

Applicants have been developing an alternative method for generating libraries of mRNA-encoded peptides, which also has the potential to work with other peptide-like polymers. In our system, a modified tRNA (tRNA^{x}) acts as a bifunctional crosslinking agent to attach an mRNA to its translation product. For an in vitro selection experiment to succeed, a molecule with some degree of the desired activity must reside in the initial population, and thus library complexity is critical. Completely in vitro methods like ours, ribosome display [7] and mRNA display [8, 9] have an advantage over methods that require the use of cells because library complexity is not limited by transformation efficiency (bacterial transformation currently limits libraries to ˜10⁹ [10]). In our system as with mRNA display, physically linking a peptide to its mRNA by covalent bonds simplifies the purification of peptide-mRNA fusions and increases the range of selectable properties because the integrity of the peptide-nucleic acid complex is not susceptible to disruption. Optimized bacterial systems are capable of translating about 10% of input mRNAs into protein [11]. Ultimately, this could increase library complexity and simplify selections by decreasing the interference from free mRNA. Other advantages of bacterial systems involve the ability to add and subtract specific components. For example, release factors could be removed and suppressor tRNAs supplied to obviate the need of removing stop codons from mRNAs [12]. Our system, as with ribosome display, uses E. coli extracts that could take advantage of these features. Here, Applicants show that the bifunctional tRNA can be used to generate 10¹¹ covalent peptide-tRNA^{x}-mRNA fusions (PTM fusions) per milliliter of translation reaction and that the fusions can be used to selectively enrich and amplify peptide libraries.

2. Results

2.1 Design and Synthesis of the Bifunctional tRNA.

Applicants designed a tRNA that can be used to form a stable linkage between polypeptides and the mRNA that encodes them. Within the ribosome, the growing peptide chain is normally linked to a tRNA by a labile ester bond. In turn, the tRNA is transiently linked to the mRNA that encodes the peptide by the base pairs of the codon-anticodon interaction. If the 3′-terminal adenosine of an aminoacyl tRNA is replaced by 3′-amino-3′-deoxyadenosine, the labile ester bond is replaced by a stable amide bond [13]. Similarly, crosslinking the wybutine base (Y base) of yeast tRNA^{phe} to the mRNA within the ribosome complex covalently joins the tRNA and mRNA [14]. Thus, a tRNA containing both an amide linkage to its amino acid and a Y base (FIG. 1A, “tRNA^{x}”) could be useful for crosslinking proteins to their encoding mRNAs. Yeast tRNA^{phe}, which contains the Y base, was purified by benzoylated DEAE cellulose chromatography, and tRNA missing its 3′-terminal adenosine was repaired with tRNA nucleotidyl transferase and 3′-amino-3′-deoxyadenosine triphosphate (FIG. 1B, lanes 1-4). Several methods exist for removing the 3′-terminal adenosine of a tRNA, but with yeast tRNA^{phe} this is generally unnecessary because the nucleotide is lost during purification [15]. Once repaired, the modified tRNA contained an intact acceptor stem with a 3′-terminal amine, and it contained the naturally occurring Y base in the anticodon loop. When the tRNA is aminoacylated with phenylalanine, the amino acid migrates to the 3′-amine forming the desired amide bond [13]. The tRNA purification, repair and aminoacylation steps were all efficient (FIG. 1B), and it was not necessary to purify the intermediates or product.

2.2. Synthesis of PTM Fusions.

PTM fusions were made by translating synthetic mRNAs in the presence of tRNA^{x} and subsequently irradiating the translation products with UV light. In the translation mix, protein synthesis proceeded normally until a phenylalanine codon (Phe codon) in the mRNA reached the A site of the ribosome. At this point, either tRNA^{x} or E. coli tRNA^{phe} could be incorporated. If tRNA^{x} was selected, it was attached to the translated peptide chain (formyl-[³⁵]-MKDYKDDDDK) (FIG. 2). No fusion products were formed if the mRNA did not code for tRNA^{x} (FIG. 2, lane 1). If normal tRNA^{phe} was selected at a Phe codon, translation continued, as shown by the production of multiple peptide-tRNA fusions (PT fusions) with an mRNA that contained multiple Phe codons (FIG. 2, lane 2). Because the number of PT fusions produced was always equal to the number of in-frame Phe codons in the mRNA, the process was likely the result of normal translation (FIG. 2, lanes 2-5). Furthermore, the mobilities of PT fusions in acrylamide gels were consistent with the size and charge of the polypeptide encoded by the translated mRNA (FIG. 2, lanes 2-5).

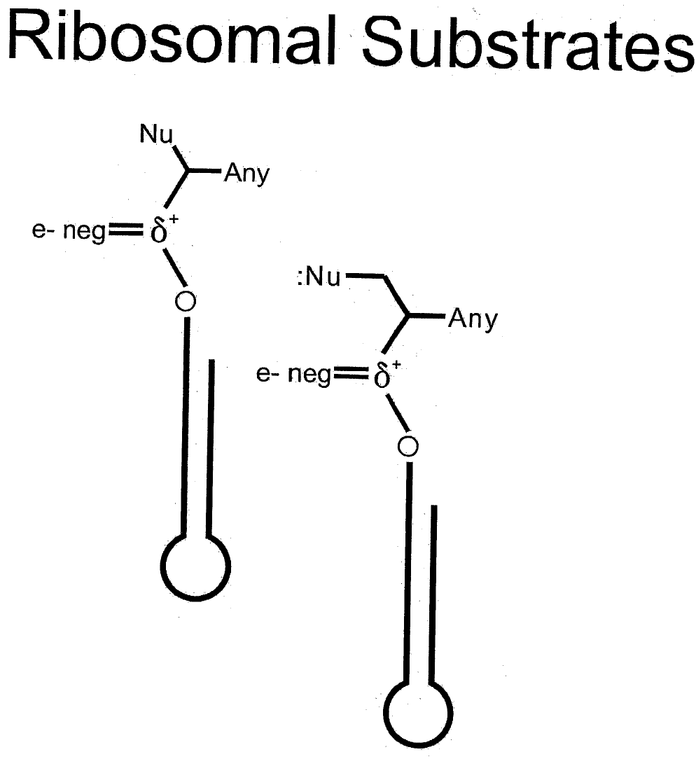

Once linked to the peptide chain, tRNA^{x} stalls protein synthesis because the ribosome cannot break the amide bond that connects it to its amino acid [13]. The stalled ribosomal complexes were stable on sucrose gradients (data not shown). Therefore, high-salt sucrose cushions were used to purify ribosomal complexes from free mRNA and ribonucleases. The ribosome-bound PT fusions were then linked to their mRNAs by UV irradiation (FIG. 3A), which crosslinks the wybutine base in tRNA^{x} to the 5′-U of the Phe codon [16] (FIG. 3B). As expected from a process that links a peptide to its mRNA, the product detected when the peptide was labeled comigrated with the product detected when mRNA was labeled (FIG. 3A, lanes 4 and 5, respectively). Controls showed that crosslinking required Phe codons in the mRNA to recruit tRNA^{x} (FIG. 3A, lanes 1 and 8), inclusion of tRNA^{x} in the translation mix (FIG. 3A, lanes 2 and 7), and exposure to UV light (FIG. 3A, lanes 3 and 6). Quantitation of the PT and PTM fusion bands (FIG. 3A, lane 4) indicated that about 0.2% of the mRNA was decoded by tRNA^{x} and that 1% of the PT fusions formed PTM fusions. Although the bulk of input mRNA is degraded by contaminating ribonucleases, 10¹¹ PTM fusions were made in a 1-ml translation reaction. These results open the prospect of using tRNA^{x} to make complex pools of mRNA-encoded polypeptides.

2.3. Enrichment of Mixed Populations by Peptide Selection.

Mock in vitro selections were performed to show that PTM fusions could be used to enrich RNA sequences that encode peptides with specified properties. In the first experiment, a synthetic mRNA coding for a Cys-containing peptide and an mRNA coding for 6 consecutive histidines (poly-His) were mixed at a ratio of 1:10. The mRNA mixture was added to a translation reaction that contained tRNA^{x}, and translated peptides were crosslinked to their encoding mRNAs. PTM fusions were then removed from the ribosome and partially purified by urea-LiCl precipitation. To selectively isolate PTM fusions that contained Cys, the purified translation reaction was subjected to thiol-affinity chromatography (FIG. 4A). Quantitation of the band intensities from the initial (FIG. 4A, lane 1) and selected populations (FIG. 4A, lane 3), indicated that the initially under-represented Cys fusion was enriched about 15-fold. To control for inadvertent skewing of the makeup of the mixed population by mechanisms other than peptide selection, Applicants performed the inverse experiment; the mRNA ratio was switched and used to generate a second population of PTM fusions which were subjected to metal-affinity chromatography. Again, the initially under-represented fusion—in this case the one containing poly-His—was enriched, but by about 5-fold (FIG. 4A, lanes 4 and 6).

To show that peptide selection was reflected at the genetic level, the mRNAs contained in the initial and enriched populations were subjected to RT-PCR and compared (FIG. 4B). When thiol-affinity chromatography was used as the selective step, the PCR product encoding Cys was enriched (FIG. 4B, lanes 1-3), whereas when metal-affinity chromatography was used, the PCR product encoding poly-His was enriched (FIG. 4B, lanes 4-6). Thus, the intended selective step drives the evolution of the nucleic acid sequences that encode PTM fusions; if another mechanism was dominant—such as preferential RT-PCR amplification of a specific template—the same species would have overtaken both populations. Because RT-PCR products could be used to produce a new population of fusions, selection and amplification could be repeated to provide exponential enrichment of target molecules.

3. Discussion

By fusing a peptide to its mRNA, tRNA^{x} linked corresponding functional and replicable sequences in a single molecule. Two simple libraries of mRNAs were translated and fused to their peptide products, and the mRNAs coding for the selected peptide were amplified. These results indicate that the system can be used for the in vitro selection of peptides and proteins from complex libraries.

Library production requires the translation of mRNA pools that contain randomized coding regions. Phe codons within the randomized region could recruit tRNA^{x} early, which would produce truncated peptides. However, selection of a normal tRNA^{phe} at a Phe codon allows translation to proceed. Thus, by adding tRNA^{x} at a low effective concentration and placing a large number of Phe codons after the randomized region, most fusions will be formed near the end of an mRNA. Another concern is the presence of stop codons in the randomized region. Of the existing methods for dealing with this problem, perhaps the easiest is to use translation mixes that contain suppressor tRNAs but no release factors [17, 18]. Suppressor tRNAs that are chemically misacylated would have the added advantage of allowing the introduction of unnatural amino acids and other monomers [19, 20].

In a selection experiment, a large population is critical because it increases the likelihood that desirable molecules are represented. Currently, Applicants can make 10¹¹ fusions in a 1-ml translation reaction, which already surpasses the complexity achieved by in vivo methods. Furthermore, up to a 1000-fold improvement in fusion efficiency might be possible; if fully realized, a 1-ml reaction would yield 10¹⁴ PTM fusions. The bulk of this anticipated increase comes from improving crosslinking efficiency and increased utilization of the mRNA. For example, only 0.2 percent of the mRNA was translated and decoded by tRNA^{x} in our experiments, whereas in experiments using more highly purified bacterial translation systems over 10 percent of the mRNA is utilized for protein synthesis, perhaps because purified systems have less ribonuclease contamination [11]. If still larger libraries are desired, bacterial translation extracts can be scaled up without undue expense.

In conjunction with a more highly purified translation system, our method might offer advantages for constructing libraries synthesized from unusual monomers. Although all of the systems have the potential to incorporate unnatural amino acids by nonsense suppression, incorporating unusual monomers at sense codons is difficult in most other systems. The cognate tRNAs would need to be specifically eliminated, perhaps by use of antisense oligonucleotides, and these tRNAs would need to be replaced with “orthogonal” tRNAs that are designed to avoid editing or charging by aminoacyl-tRNA synthetases [20]. With our system, such measures would not be necessary or would be more easily accomplished because bacterial translation systems are more readily customized. For example, fusions bearing nonstandard polymers have been generated in translation mixes that use misacylated tRNA, EFG and EFTu rather than total tRNA and S150 (data not shown). In principal, ribosome display and mRNA display could use similar translation systems. However, mRNA display has not been shown to work with bacterial ribosomes, and ribosome display requires the translation of much longer peptides, as over 40 residues must be translated before the peptide begins to emerge from the exit channel of the ribosome [7]. With PTM fusions, even short open reading frames can satisfy the requirements of complexity and accessibility. Thus, because the ribosome can use hundreds of monomers [e.g., 21-24], it could be possible to build low-molecular-weight libraries that have desirable properties such as protease resistance, permeability, and conformational rigidity. In conjunction with in vitro selection methods, such libraries could open the door to a vast array of useful molecules that could serve as leads for the development of therapeutics and other useful reagents.

4. Significance

Applicants anticipate that the flexibility of our system with respect to the types of polymers that could be produced will broaden the number of applications to which in vitro selection can be applied. In principal, fusion libraries that are larger than 10¹¹ could be constructed from any combination of monomers that the ribosome can polymerize. This large complexity has the potential to generate rare-functional molecules, while the wide range of acceptable monomers could be used to adjust the overall physical and chemical properties of a library. Thus, the method could be useful for evolving non-biological polymers whose properties depart from those accessible to peptides and proteins.

5. Materials and Methods

5.1. Purification of Ribosomes and S150 Enzyme Fraction.

For ribosomes, 5 g of an E. coli ribonuclease-deficient strain (A19) were washed with 300 ml of buffer A (10 mM Tris-HCl, pH 7.5, 10 mM Mg-acetate, 22 mM NH₄Cl, 1 mM DTT), pelleted in a Sorvall SLA-3000 rotor (4600 g), and suspended in buffer A in a final volume of 20 ml. The suspension was lysed in a BeadBeater mixer (Biospec) according to the manufacturers directions with 80 ml of 0.1 mm zirconia/silica beads. The beads were washed several times with buffer A and the supernatants combined and transferred to 13 ml centrifuge tubes. The lysate was cleared by repeated 20 min centrifugations in a Sorvall SS-34 rotor (17,000 g). Ribosomes were isolated by layering 13 ml of the clarified supernatant on 13 ml of 32% sucrose in buffer A and centrifuging for 13 hours in a Beckman 70Ti rotor (120,000 g). Pellets were dissolved in a small volume of buffer A, and the concentration was adjusted to 45 μM before storage at −80° C. in 5-25 μl aliquots. For the S150 enzyme fraction, 4 g of cells were washed in 38 ml of buffer B (10 mM Tris-HCl, pH 7.5, 10 mM MgCl₂, 30 mM NH₄Cl, 6 mM BME) and lysed as before but with buffer B. The lysate was clarified twice by centrifugation for 20 min in a Sorvall SS-34 rotor (30,000 g) and once for 30 min in a Beckman VTi50 rotor (150,000 g). The clarified supernatant was loaded on a 40 ml DEAE sepharose column (Pharmacia) that had been equilibrated with buffer B, and the column was washed with 1 L of buffer B. The S150 enzyme fraction was eluted with buffer B plus 220 mM NH₄Cl. Fractions were examined by eye, and the dark brown ones were pooled and stored at −80° C. in 20-100 μl aliquots.

5.2. Synthesis of the Bi-Functional tRNA^{x}.

Yeast tRNA^{phe} was purified by benzoylated DEAE cellulose chromatography [25]. 3′-Amino-3′-deoxyadenosine triphosphate was prepared by published protocols [26, 27]. The construct pQECCA, expressing E. coli tRNA nucleotidyl transferase as a fusion protein with a poly-His tag, was a generous gift from U. RajBhandary (MIT). Yeast tRNA^{phe} missing its 3′-terminal adenosine was repaired by incubating 500 μM tRNA, 2 mM 3′-amino-3′-deoxyadenosine triphosphate, and tRNA nucleotidyl transferase in buffer (50 mM Tris-HCl, pH 8.0, 10 mM MgCl₂, 30 mM KCl, 5 mM DTT, 0.5 mg/ml BSA) for 10 min at 37° C. Protein was removed by phenol extraction, and the tRNA was ethanol precipitated. The intermediate (3′-amino-tRNA^{phe}) was charged by incubating 25 μM 3′-amino-tRNA^{phe}, 100 μM phenylalanine, 4 mM dATP, with 1/5 (vol/vol) S150 enzyme fraction for 30 min at 37° C. in 30 mM Tris-HCl, pH 7.5, 15 mM MgCl₂, 25 mM KCl, and 5 mM DTT. The final product (tRNA^{x}) was purified by phenol extraction and ethanol precipitated.

5.3. Synthesis of mRNA.

DNA templates for T7 in vitro transcription were generated by PCR, using appropriate templates and primers. PCR products were ethanol precipitated and transcribed in half their original volume (40 mM Tris-HCl, pH 7.9, 26 mM MgCl₂, 2.5 mM spermidine, 0.01% triton X-100, 5 mM ATP, 5 mM CTP, 8 mM GTP, 2 mM UTP, and T7 RNA polymerase). All mRNAs (Table 1) were gel-purified.

5.4. Synthesis of PT Fusions.

Three μg of mRNA was translated in 40 μl of an E. coli S30 extract (Promega) according to the manufacturers protocol. In addition to exogenous mRNA, reactions contained [³⁵S]-methionine and 2 μM tRNA^{x}. After incubating for 30 min at 37° C., the reactions were terminated by phenol extraction and ethanol precipitated. Amino acids and peptides bound to normal tRNAs were removed by incubation in 0.5 M Ches-KOH, pH 9.5 for 1 hr at 37° C., followed by phenol extraction and ethanol precipitation.

5.5. Synthesis of PTM Fusions.

For each reaction, translation premix contained 2.5 μl of 10× translation buffer (500 mM Tris-acetate, pH 8.0, 110 mM Mg-acetate, 1 M NH₄Cl, 10 mM DTT); 2.5 μl of 100 mM phosphoenol pyruvate; 0.5 μl of 100 mM ATP: 10 mM GTP; 0.5 μl of 25 μg/μl total E. coli tRNA; 2.5 μl of 1 mM amino acids; 0.5 μl of 1 μg/μl pyruvate kinase; 3 μl of 18 μM ribosomes; and 6 μl of S150 enzyme fraction. Premix was incubated for 10 min at 37° C. after which 2.5 μl of 200 μM tRNA^{x} was added when appropriate. Aliquots (16 μl) of the premix were distributed among tubes that contained 2 μl of [³⁵S]-methionine labeled fMet-tRNA^{Met} and 2 μl of 50 μM mRNA. When the mRNA rather than the peptide was labeled, the tubes contained 2 μl of fMet-tRNA^{Met} and 2 μl of 50 μM [³²P]-cordycepin-labeled mRNA. Incubation was continued for another 10 min and samples layered onto 500 μl sucrose cushions (32% sucrose, 50 mM Hepes, pH 7.8, 20 mM MgCl₂, 500 mM NH₄Cl, 6 mM BME). Stalled ribosomal complexes were pelleted by centrifugation for 45 min at 4° C. in a Beckman TLA 100.2 rotor (360,000 g). Pellets were dissolved in 25 μl of buffer (40 mM Hepes, pH 7.8, 20 mM MgCl₂, 80 mM NH₄Cl, 6 mM BME), spotted on a polystyrene petri-dish, covered with the supplied lid and exposed to UV for 20 min using a 450 watt Hanovia bulb with water jacket (Ace Glass). As before, phenol extraction and base treatment were used to remove undesired translation products.

5.6. In Vitro Peptide Selection.

PTM fusions were synthesized as described above with minor modifications. Briefly, [³⁵S]-methionine labeled fMet-tRNA^{Met} was generated in situ by adding 1/10 (vol/vol) deprotected 5,10-methenyltetrahydrofolate [28] and 1/10 (vol/vol) [³⁵S]-methionine; the amino acid mix did not contain methionine. To make room for the additional reagents, half as much amino acid mix was added, concentrated stocks of nucleotide mix and ribosomes were used, and the final concentration of tRNA^{x} was reduced to 10 μM. Rather than individual mRNAs, mixtures were used to direct translation; mRNA encoding the target peptide was at 0.4 μM and mRNA encoding the background peptide was at 4 μM. After crosslinking, ribosomal complexes were mixed 1:1 with lithium buffer (8 M urea, 4 M LiCl, 10 mM EDTA, pH 8.0, 6 mM BME) and precipitated overnight at 4° C. Pellets were dissolved in 20 mM Tris-HCl, pH 7.5, 2 mM MgCl₂, 60 mM KCl, 6 mM BME and stored at −20° C. Before selection, aliquots were precipitated from ethanol and dried by aspiration to remove BME, then dissolved in 10 μl of water. Poly-His-containing fusions from a 90 μl translation reaction were bound to Talon metal-affinity resin (Clontech) by adding 9 μl of the concentrated product to a resin slurry (100 μl resin: 100 μl 50 mM Tris-HCl, pH 7.5, 300 mM NH₄Cl, 0.25 mg/ml BSA) and rotating for 16 hr at 4° C. Unbound fusions were removed by washing the resin 5 times with 100 μl aliquots of wash buffer (50 mM Tris-HCl, pH 7.5, 1 M NH₄Cl). Bound fusions were eluted by washing the resin 3 times with 100 μl of wash buffer plus 10 mM EDTA, pH 8.0, and 8.3 μg of total E. coli tRNA. Cys-containing fusions were immobilized in a similar manner with 500 μl of activated Thiol-Sepharose 4B (Pharmacia) in 5 ml of buffer (25 mM Tris-HCl, pH 7.5, 300 mM NaCl, 7M urea). After binding, the resin was poured into a column and washed with 100 ml of the same buffer. The resin was then removed and eluted 3 times with 1 ml aliquots of buffer that also contained 50 mM DTT and 25 μg/ml total E. coli tRNA. Column washes and eluants were ethanol precipitated and dissolved in 90 μl or 9 μl of water, respectively. For both selections, before RT-PCR, unfused mRNA was removed from the enriched population of PTM fusions by gel purification; a liberal section of the gel was excised to insure that gel purification did not influence the ratio of the two PTM fusions.

TABLE I

Sequences of the mRNAs used to direct in vitro
protein synthesis.

| | |
|---|---|
| mRNA | Sequence |

| | |
|---|---|
| 1 | GGAUCCUAGGAAGCUUGAAGGAGAUAUACCAUG AAA GAC UAC AAG |
| | GAC GAC GAC GAC AAG UAU AAA GUU . . . |
| 2 | GGAUCCUAGGAAGCUUGAAGGAGAUAUACCAUG AAA GAC UAC AAG |
| | GAC GAC GAC GAC AAG UUU UUU UUU . . . |
| 3 | GGAUCCUAGGAAGCUUGAAGGAGAUAUAGGAUG AAA GAC UAC AAG |
| | GAC GAC GAC GAC AAG UUU AAA GUU . . . |
| 4 | GGAUCCUAGGAAGCUUGAAGGAGAUAUACCAUG AAA GAC UAC AAG |
| | GAC GAC GAC GAC AAG UAU UUU GUU . . . |
| 5 | GGAUCCUAGGAAGCUUGAAGGAGAUAUACGAUG AAA GAC UAC AAG |
| | GAC GAC GAC GAC AAG UAU AAA UUU . . . |
| 6 | GGGUUAACUUUAGAAGGAGGUAAAAAAAAUG AAA CGU GAA AAG |
| | ACA UUU UUU UUU |
| 7 | GGGUUAACUUUAGAAGGAGGUAAAAAAAAUG AAA CGU GAA AAG |
| | ACA GAA CGU ACA |
| 8 | GGGUUAACUUUAGAAGGAGGUAAAAAAAAUG AAA CAC CAU CAC |
| | CAC CAU CAC GGA AAU CGU UUU UUC UUU UUC UUU UUC CGC UAG |
| | CGU CAG GGC UAU UCA CCA UUA ACC CAC UAG GGC GUU |
| 9 | GGGUUAAGUUUAGAAGGAGGUAAAAAAAAUG CGU UGC GAU CAC |
| | GGA AAU CGU UUU UUC UUU UUC UUU UUC CGC UAG CGU CAG GGC |
| | UAU UCA CCA UUA ACC CAC UAG GGC GUU |
| 10 | GGGUUAACUUUAGAAGGAGGUAAAAAAAAUG UUU AAA GAA AAG |
| | UUU GAA CGU ACA |

For each mRNA, the codons are underlined and the initiator methionine codon (AUG) and Phe codons (UUU or UUC) are in bold. For mRNAs 1-5, part of the sequence is not shown ( . . . ).
The remaining sequence is free of phenylalanine codons and is the same as nucleotides 217-444 of beta-lactamase.

The present invention is further illustrated by the following examples, which are not intended to be limiting in any way.

### EXAMPLE 1

### Crosslinking of Yeast tRNA^{phe} and Peptidyl Analogues Yeast tRNA^{phe} to mRNA

Ribosome Preparation

Tight couple ribosomes were prepared from Escherichia (E.) coli strain MRE 600. Two liters of LB broth were innoculated with 20 ml of a fresh overnight culture and incubated at 37° C. with vigorous agitation until an absorbance of 0.6 (550 nm) was reached. Culture flasks were then transferred to an ice water bath and incubated for 30 minutes. Cells were pelleted by centrifugation at 8000 rpm in a Sorvall GSA rotor, for 10 minutes, at 4° C. Cells were washed by resuspension in approximately 50 ml of buffer A (50 mM Tris-HCl, pH 7.5; 10 mM MgCl₂; 100 mM NH₄Cl, 6 mM 2-mercaptoethanol, 0.5 mM EDTA) and pelleted by centrifugation for 5 minutes at 8000 rpm in a Sorvall SS-34 rotor. The pelleted cells were then transferred to a large mortar at 4° C. Lysis of cells was performed in the 4° C. cold room by slow addition of 4° C. alumina (2.5 grams for every gram of cells) while grinding with a pestle. Typically, lysis took about twenty minutes. When lysis was complete, buffer A was added to dilute the grinding mixture to a total volume of about 30 ml. This was divided evenly between two Sorval SS-34 centrifuge tubes and the alumina was cleared from the solution by centrifugation for 5 minutes at 5000 rpm. The supernatant (cell lysate) was removed and clarified from cellular debris by centrifugation for 20 minutes at 15,000 rpm in a Sorvall SS-34 rotor; the clarification was then repeated. The NH₄Cl concentration of the clarified supernatant was adjusted to 500 mM and the ribosomes were pelleted by centrifugation for 4 hours, at 4° C., at 40,000 rpm in a Beckman Ti60 rotor. The ribosome pellet was washed with approximately 5 ml of buffer B (50 mM Tris-HCl, pH 7.5; 6 mM MgCl₂; 100 mM NH₄Cl, 6 mM 2-mercaptoethanol, 0.5 mM EDTA) and then resuspended in 4 ml of buffer B by adding a small stir bar directly to the centrifuge tube and mounting the tube above a stir plate in the 4° C. coldroom. (It is important to maintain gentle stirring throughout resuspension of the ribosomes.) When the ribosomes were completely resuspended (20-60 minutes) 1 ml aliquots were layered onto 36 ml, 10-50% sucrose gradients (in buffer B) and centrifuged for 13 hours, at 4° C., at 20,000 rpm in a Beckman SW28 rotor. The gradients were fractionated and the 70S ribosome peak was collected. The magnesium concentration was adjusted to 10 mM and the ribosomes were pelleted from solution by centrifugation for 4 hours, at 4° C., at 60,000 rpm in a Beckman Ti60 rotor. The ribosome pellet was washed with 5 ml of buffer C (50 mM Tris-HCl, pH 7.5; 6 mM MgCl₂; 100 mM NH₄Cl, 0.5 mM EDTA) and resuspended as before in 1 ml of buffer C. The concentration of the ribosomes was determined at 260 nm (1 O.D. of 70S ribosomes=23 pmol) and buffer C was added to obtain a final concentration of 10 μM. The ribosomes were then aliquoted (10 μl), frozen in liquid nitrogen and stored at −70° C.

Preparation of S100 Extract

Twenty grams of E. coli cells were lysed and the cellular debris removed as described above. Ribosomes were removed from the clarified cell lysate by centrifugation for 4 hours, at 4° C., at 40,000 rpm in a Beckman Ti60 rotor and the supernatant was diluted two fold with buffer D (10 mM Tris-HCl pH 7.5; 30 mM NH₄Cl; 10 mM MgCl₂; and 6 mM 2-mercaptoethanol). This solution was stirred with 12 g of dry DEAE-cellulose that was equilibrated with buffer D, washed with distilled water, and then dried. The slurry was filtered on a scintered glass funnel and washed with 1-2 liters of buffer D. The DEAE-cellulose cake was resuspended in buffer D and packed in a column. The S100 extract was then eluted with buffer D containing 250 mM NH₄Cl (the desired fractions elute as a sharp band and can usually be identified by eye as they have a pale yellow color. Small aliquots (100 μl) of the purified S100 extract were frozen in liquid nitrogen and stored at −70° C. The tRNA charging efficiency of S100 was determined by monitoring TCA-precipitable counts in the presence of tRNA^{phe} and radiolabeled phenylalanine [S100 is incubated for 10 minutes at 37° C. with tRNA^{phe} (1 μM), 14C-phenylalanine (20 μM), and ATP (2 mM) in 30 mM Tris-HCl pH 7.5, 15 mM 1M MgCl₂, 25 mM KCl, 4 mM DTT].

Preparation of tRNAs and mRNA

Yeast tRNA^{phe} was purchased from Sigma. Elongator methionine tRNA was transcribed by T7 polymerase from DNA obtained by PCR amplification of E. coli chromosomal DNA using the (+) strand primer TAA-TAC-GAC-TCA-CTA-TAG-GCT-ACG-TAG-CTC-AGT-TGG (SEQ ID NO.: 1) and the (−) strand primer TGG-TGG-CTA-CGA-CGG-GAT-TC (SEQ ID NO.: 2). A portion of the T4 gene-32 mRNA was transcribed by T7 polymerase from a PCR amplified section (−55 to +85) of plasmid pKsKl2A (Krisch and Allet, PNAS (USA), 79:4937-4941 (1982)) using the (+) strand primer TAA-TAC-GAC-TCA-CTA-TAG-GTA-AAG-TGT-CAT-TAG-C (SEQ ID NO.: 3)and the (−) strand primer CTT-TAT-CTT-CAG-AAG-AAA-AAC-C (SEQ ID NO.: 4). The mRNA was labeled with yeast polyA polymerase and ³²P labeled cordycepin.

Amino Acylation and Acetylation of Yeast tRNA^{phe}

Amino acylation of yeast tRNA^{phe} (1 μM) with phenylalanine (20 μM) was performed by incubation for 10 minutes at 37° C. with ATP (2 mM) and the appropriate amount of S100 in 30 mM Tris-HCl pH 7.5, 15 mM 1M MgCl₂, 25 mM KCl, 4 mM DTT. To stop the reaction and recover the acylated tRNA (PHE-tRNA^{phe}; typically 70% yield) one-tenth volume of 3M NaOAc pH 5.0 was added, and the reaction was extracted two times with acid phenol, two times with chloroform, and precipitated with 2.5 volumes of ethanol. The PHE-tRNA^{phe} was resuspended at a concentration of 10 μM in 0.2M NaOAc pH 5.0 and the amine of the phenylalanine was acetylated (to produce the peptidyl analogue N-acetyl-PHE-tRNA^{phe}) by adding one-hundredth volume of acetic anhydride followed by incubation on ice for 30 minutes. A fresh aliquot of acetic anhydride was added and the incubation continued for another 30 minutes. The reaction was stopped and the N-acetyl-PHE-tRNA^{phe} isolated by adding 2.5 volumes of ethanol and precipitating. N-acetyl-PHE-tRNA^{phe} was resuspended at a concentration of 10 μM in 10 mM NaOAc pH 5.0 and stored at −20 μC.

Binding of mRNA and tRNA to the Ribosome and Crosslinking of N-acetyl-PHE-tRNAphe to the mRNA

Ribosomal complexes with tRNA^{met} bound to the P site of the ribosome and the first codon (AUG) of the labeled gene-32 mRNA were formed by incubating ribosomes (0.5 μM), tRNA^{met} (1 μM), and mRNA (5 μM) in 25 μl of buffer (Tris-HCl ph 7.5, 30 mM NH₄Cl, 20 mM MgCl₂) for 10 minutes at 37° C. Deacylated yeast tRNA^{phe} was bound to the A site (corresponding to the second codon (UUU) of the mRNA) by adding 25 pmol of the tRNA and continuing the incubation for an additional 10 minutes. Alternatively, N-acetyl-PHE-tRNA^{phe} was bound to the P site by adding 25 pmol of N-acetyl-PHE-tRNA^{phe} and continuing the incubation for an additional 10 minutes; the tRNA initially binds to the A site but spontaneous translocation should move it into the P site of the ribosome. The phenylalanine tRNA in either type of complex was crosslinked to the mRNA by spotting the reaction on a petri dish and irradiating for 45 minutes (at a distance of 10 cm) with a 450 watt, medium pressure, mercury vapor lamp (ACE glass). The samples were maintained at 4° C. during the irradiation and short wavelength UV light was blocked by a suitable filter (the top of a plastic petri dish). The extent of crosslinking between the mRNA and tRNA (typically C20% of the ribosomes contain crosslinked, tRNA-mRNA fusions) was determined by polyacrylamide gel electrophoresis.

### EXAMPLE 2

### Synthesis of a Bifunctional tRNA and Its Use in Generating Encoded Peptide Libraries

PHE-N-tRNA

A method to produce PHE-N-tRNA involves replacement of the 3′-terminal A of a tRNA with 3′-amino-3′-deoxyadenosine and charging of the 3′-amino-3′-deoxyadenosine substituted tRNA with phenylalanine (Fraser, T. H. and Rich, A., Proc. Nat. Acad. Sci. USA 70:2671 (1972)). Using this procedure with yeast tRNA^{phe} generates PHE-N-tRNA, a tRNA analogue that can act as an acceptor substrate but not a donor substrate during protein synthesis and that can be crosslinked to mRNA. First, the 3′-terminal A, of yeast tRNA^{phe} is removed (resulting in tRNA(−A)) and then 3′-amino-3′-deoxyadenosine monophosphate is attached to the end of tRNA(−A) by the action of tRNA nucleotidyl transferase. This 3′-amino-3′-deoxyadenosine substituted tRNA is then charged with phenylalanine and S100.

Strategy for Synthesis of a Bifunctional tRNA (PURO-tRNA^{phe})

A method to produce PURO-tRNA (a tRNA with its 3′ terminal A residue replaced by puromycin) involves replacement of the 3′-terminal C and A of a tRNA with pCpPuromycin (puromycin that has been extended at its 5′ hydroxyl by a 3′,5-cytidine diphosphate). The 3′-terminal C and A of yeast tRNAphe are removed (resulting in tRNA(−CA)) and then pCpPuromycin is ligated to the end of the truncated tRNA to seamlessly fuse the two molecules. Using this procedure with yeast tRNA^{phe} generates PURO-tRNA^{phe}, a tRNA analogue that can act as an acceptor substrate but not a donor substrate during protein synthesis and that can be crosslinked to mRNA.

Removal of the 3′-Terminal A and Removal of the 3′-Terminal C and A of Yeast tRNA^{phe}

Yeast tRNA^{phe} (300 μM) and venom phosphodiesterase (Crotalus atrox) were incubated in 40 mM glycine-NaOH pH 8.7, and 10 mM magnesium acetate for 10 minutes at 37° C. Typically, 0.1 to 10 mg of venom per ml have been used in the literature; however, it was useful to titrate the venom to identify the concentration that produced quantitative removal of the C and A. After incubation, the reaction was brought to 0.3 M sodium acetate, 5 mM EDTA, 0.5% SDS, phenol extracted three times, chloroform extracted two times, 2.5 volumes of ethanol were added and the tRNA was precipitated. If tRNA (−A) was the desired product the tRNA was incubated with tRNA nucleotidyl transferase and CTP in 50 mM Glycine pH 9.2, 30 mM KCl, 12.5 mM MgCl₂, 2.5 mg/ml reduced glutathione and 0.375 mg/ml BSA for 30 minutes. The reaction was phenol extracted and tRNA(−A) resuspended in water. If the desired product was tRNA(−CA) the tRNA was purified by polyacrylamide gel electrophoresis and was resuspended in water and then adjusted to a concentration of 10 μM in 70 mM Tris-HCl pH 8.5, 30 mM MgCl2, and 1.6 mM DTT.

Synthesis of 3′-amino-3′-deoxyadenosine and Attachment to tRNA(−A).

3′-amino-3′-deoxyadenosine was made by the method of Gerber and Lechevalier (J. Org. Chem. 27:1731(1962)) and can be phosphorylated according to Fraser and Rich (Proc. Nat. Acad. Sci. USA 70:2671 (1973)). 3′-amino-3′-deoxyadenosine triphosphate, tRNA(−A), and tRNA nucleotidyl transferase were used to generate yeast N-tRNA (yeast tRNA^{phe} whose 3′-terminal adenosine has been substituted with 3′-amino-3′-deoxyadenosine) by incubation at 37° C. for 30 minutes in 50 mM Glycine pH 9.2, 30 mM KCl, 12.5 mM MgCl₂, 2.5 mg/ml reduced glutathione and 0.375 mg/ml BSA. PHE-N-tRNA was generated by incubating N-tRNA, phenylalanine and S100 at 37° C. for 30 minutes in 30 mM Tris pH 7.5, 25 mM KCl, 15 mM MgCl₂, and 4 mM DTT. PHE-N-tRNA was purified by HPLC.

Synthesis of pCpPuromycin and Attachment to the Truncated tRNA

Several methods have been used to make CpPuromycin and CpPuromycin analogues (e.g. Harris, R. J. et al., Can. J. Bioch., 50:918-926(1972); Nyilas, A. et al., Bioorganic and Medicinal Chemistry Letters, 3(6):1371-1374(1993); Green and Noller, Science, 1998). CpPuromycin prepared by the method of Green and Noller (1998) is used. CpPuromycin (10 μM) is phosphorylated by incubation at 37° C. for 30 minutes with T4 polynucleotide kinase in 70 mM Tris-HCl pH 7.5, 10 mM MgCl₂, 5 mM DTT, and 30 μM ATP. (It is important to keep the concentration of ATP at 2-5 fold over the concentration of CpPuromycin as the subsequent ligation is sensitive to excessive ATP). The kinase is inactivated by heating the reaction to 65° C. for 20 minutes. The solution is brought to 20 μg/ml BSA, an equal volume of 10 μM tRNA(−CA) in (70 mM Tris-HCl pH 8.5, 50 mM MgCl2, and 1.6 mM DTT) is added, and the solution is incubated for 60 minutes at 37° C. with T4 RNA ligase. Ligation of pCpPuromycin to tRNA(−CA) produces the desired PURO-tRNA^{phe} product which is purified by polyacrylamide gel electrophoresis.

### EXAMPLE 3

### Using the Bifunctional tRNA to Generate an Encoded Polypeptide Library

An in vitro translation mixture is combined with a complex pool of mRNA sequences, and an appropriate amount of a bifunctional tRNA (PHE-N-tRNA or PURO-tRNA^{phe}). The translation mixture contains all of the factors required for in vitro translation (e.g., initiation factors, the tRNAs, elongation factors, amino acids, etc.) except for mRNA. Translation mixtures are purchased (Promega) or they can be made from crude cellular extracts (extracts from several organisms are used and protocols for making them are readily available in the literature). The appropriate concentration of a bifuntional tRNA is added to the translation mixture. Translation is initiated by the addition of the complex pool of mRNA sequences. All of the members of the pool of mRNA sequences have a constant sequence at their 5′ end that permits them to be translated by the ribosome, an internal, randomized, polypeptide-coding segment that is devoid of stop codons, and a UUU- and UUC-rich 3′ coding segment that recruits a bifunctional tRNA after translation of the randomized segment is completed. The bifunctional tRNA concentration in the translation mixture is carefully adjusted so that during translation, the UUU and UUC codons that are contained within the randomized coding segment are decoded by normal phenylalanine tRNA that are present in the translation mixture. When the UUU- and UUC-rich 3′ coding segment (of each translating mRNA) is reached, there is a high probability that a bifunctional tRNA will eventually be selected to decode a UUU or UUC codon. Decoding by a bifunctional tRNA stalls translation of the mRNA because a bifunctional tRNA can accept the growing polypeptide chain but cannot transfer the polypeptide chain to another acylated tRNA. This generates polypeptide-bifunctional tRNA fusions that are bound to ribosomes. The translation mixture is concurrently or subsequently irradiated at a distance of 10 cm with a 450 watt, medium pressure, mercury vapor lamp (ACE glass; short wavelength UV light is blocked by a suitable filter). This activates the Y base of the bifunctional tRNA and causes the base (and therefore the polypeptide-bifunctional tRNA fusion) to become crosslinked to the mRNA that encodes the polypeptide. After irradiation, disruption of the ribosomes with EDTA releases the encoded polypeptide library so that it can be purified and used.

While this invention has been particularly shown and described with references to preferred embodiments thereof, it will be understood by those skilled in the art that various changes in form and details may be made therein without departing from the spirit and scope of the invention as defined by the appended claims. Those skilled in the art will recognize or be able to ascertain using no more than routine experimentation, many equivalents to the specific embodiments of the invention described specifically herein. Such equivalents are intended to be encompassed in the scope of the claims.

### EXAMPLE 4

### Generation of Nonstandard Polymer-tRNA Analogue-mRNA Fusions

Fusions bearing nonstandard polymers were synthesized by translating mRNAs containing open reading frames of 5 to 10 lysine codons (AAA). Mischarged tRNA was made from trans-lysine and purified lysine tRNA (Sigma) using S150 extracts and subsequent purification—the modified amino acid (trans-lysine) deceives lysine aminoacyl tRNA synthetase present in the extract and becomes attached to lysine tRNA providing a charged tRNA with a nonstandard monomer. Alternatively, Biotin-lys tRNA (Promega) was used as a source of tRNA bearing a nonstandard monomer. Translation reactions were performed with EFTu, EFG, fmet-tRNA, mRNA, ribosomes, the bifunctional tRNA and one of the above mischarged tRNAs in an appropriate composition for ribosome directed polymerization. Nonstandard polymers resulted as shown by the production of fusions that depended on the inclusion of one of the two mischarged tRNAs. Furthermore, gel shift experiments with streptavidin showed that the resulting fusions have decreased mobilities in polyacrylamide gels, demonstrating the ribosome directed polymerization of the nonstandard-biotin-containing monomer and its inclusion in nonstandard polymer-bifuntional tRNA-mRNA fusions.

### REFERENCES

1. Gold, L., Polisky, B., Uhlenbeck, O. and Yarus, M. (1995). Diversity of oligonucleotide functions. Annu. Rev. Biochem. 64, 763-797.

2. Lorsch, J. R. and Szostak, J. W. (1996). Chance and necessity in the selection of nucleic acid catalysts. Acc. Chem. Res. 29, 103-110.

3. Kurz, M. and Breaker, R. R. (1999). In vitro selection of nucleic acid enzymes. Curr. Top. Microbiol. Immunol. 243, 137-158

4. Bartel D. P. and Unrau P. J. (1999). Constructing an RNA world. Trends Cell Biol. 9, M9-M13.

5. Smith, G. P. (1985). Filamentous fusion phage: novel expression vectors that display cloned antigens on the virion surface. Science 228, 1315-1317.

6. Cull, M. G., Miller, J. F. and Schatz, P. J. (1992). Screening for receptor ligands using large libraries of peptides linked to the C terminus of the lac repressor. Proc. Natl. Acad. Sci. USA. 89, 1865-1869

7. Mattheakis, L. C., Bhatt, R. R., and Dower, W. J. (1994). An in vitro polysome display system for identifying ligands from very large peptide libraries. Proc. Natl. Acad. Sci. USA. 91, 9022-9026.

8. Nemoto, N., Miyamoto-Sato, E., Husimi, Y. and Yanagawa, H. (1997). In vitro virus: bonding of mRNA bearing puromycin at the 3′-terminal end to the C-terminal end of its encoded protein on the ribosome in vitro. FEBS Letters. 414, 405-408.

9. Roberts, R. W. and Szostak, J. W. (1997). RNA-peptide fusions for the in vitro selection of peptides and proteins. Proc. Natl. Acad. Sci. USA. 94, 12297-12302.

10. Noren, K. A. and Noren, C. J. (2001). Construction of high-complexity combinatorial phage display peptide libraries. Methods 23, 169-178.

11. Pavlov, M. Y. and Ehrenberg, M. (1996). Rate of translation of natural mRNAs in an optimized in vitro system. Arch. Biochem. Biophys. 328, 9-16.

12. Cho, G., Keefe, A. D., Liu, R., Wilson, D. S. and Szostak, J. W. (2000). Constructing high complexity synthetic libraries of long ORFs using in vitro selection. J. Mol. Biol. 297, 309-319

13. Fraser, T. H. and Rich, A. (1973). Synthesis and aminoacylation of 3′-amino-3′-deoxy transfer RNA and its activity in ribosomal protein synthesis. Proc. Natl. Acad. Sci. USA. 70, 2671-2675.

14. Matzke, A. J., Barta, A. and Kuechler, E. (1980). Mechanism of translocation: relative arrangement of tRNA and mRNA on the ribosome. Proc. Natl. Acad. Sci. USA. 77, 5110-5114.

15. RajBhandary, U. L., Stuart, A., Hoskinson, R. M. and Khorana, H. G. (1968). Studies on polynucleotides. 78. Yeast phenylalanine transfer ribonucleic acid: terminal sequences. J. Biol. Chem. 243, 565-574.

16. Steiner, G., Luhrmann, R. and Kuechler, E. (1984). Crosslinking transfer RNA and messenger RNA at the ribosomal decoding region: identification of the site of reaction on the messenger RNA. Nucleic Acids Res. 12, 1.81-191.

17. Short, G. F., Golovine, S. Y. and Hecht, S. M. (1999). Effects of release factor 1 on in vitro protein translation and the elaboration of proteins containing unnatural amino acids. Biochemistry. 38, 8808-8819.

18. Shimizu, Y., Inoue, A., Tomari, Y., Suzuki, T., Yokogawa, T., Nishikawa, K. and Ueda, T. (2001). Cell-free translation reconstituted with purified components. Nat. Biotechnol. 19, 751-755.

19. Heckler, T. G., Chang, L. H., Zama, Y., Naka, T., Chorghade, M. S. and Hecht, S. M. (1984). T4 RNA ligase mediated preparation of novel “chemically misacylated” tRNAPheS. Biochemistry. 23, 1468-1473.

20. Noren, C. J., Anthony-Cahill, S. J., Griffith, M. C. and Schultz, P. G. (1989). A general method for site-specific incorporation of unnatural amino acids into proteins. Science 244, 182-188.

21. Bain, J. D., Wacker, D. A., Kuo, E. E. and Chamberlin, A. R. (1991). Site-specific incorporation of normatural residues into peptides—effect of residue structure on suppression and translation efficiencies Tetrahedron 47, 2389-2400.

22. Mendel, D., Ellman, J. and Schultz, P. G. (1993). Protein-biosynthesis with conformationally restricted amino-acids. J. Am. Chem. Soc. 115, 4359-4360.

23. Thorson, J. S., Cornish, V. W., Barrett, J. E., Cload, S. T., Yano, T. and Schultz, P. G. (1998). A biosynthetic approach for the incorporation of unnatural amino acids into proteins. Meth. Mol. Biol. 77, 43-73.

24. Hohsaka, T., Kajihara, D., Ashizuka, Y., Murakami, H. and Sisido, M. (1999). Efficient incorporation of normatural amino acids with large aromatic groups into streptavidin in in vitro protein synthesizing systems. J. Am. Chem. Soc. 121, 34-40.

25. Gillam, I., Millward, S., Blew, D., von Tigerstrom, M., Wimmer, E., and Tener, G. M. (1967). The separation of soluble ribonucleic acids on benzoylated diethylaminoethylcellulose. Biochemistry. 6, 3043-3056.

26. Robins, M. J., Hawrelak, S. D., Hernandez, A. E. and Wnuk, S. F. (1992). Nucleic-acid related-compounds. 71. Efficient general-synthesis of purine (amino, azido, and triflate)-sugar nucleosides. Nucleosides and Nucleotides. 11, 821-834.

27. Morr, M. and Wray, V. (1994). New cyclic-derivatives of 3′-amino-3′-deoxyadenosine-5′-diphosphate, 3′-amino-3′-deoxyadenosine-5′-triphosphate, and 3′-amino-3′-deoxyadenosine-5′-methylenebis(phosphonate). Angew. Chem. Int. Ed. 33, 1394-1396.

28. Blanquet, S., Dessen, P. and Kahn, D. (1984). Properties and specificity of methionyl-tRNAfMet formyltransferase from Escherichia coli. Meth. in Enz. 106, 141-152.

29. Matzke, A. J., Barta, A. and Kuechler, E. (1980). Photo-induced crosslinking between phenylalanine transfer RNA and messenger RNA on the Escherichia coli ribosome. Eur. J. Biochem. 112, 169-178.

What is claimed is:

1. A tRNA analogue, comprising:
(a) a tRNA;
(b) an amino acid moiety which acts as an acceptor substrate, but not as a donor substrate, during translation; and
(c) a reactive or activatible moiety near or within the anticodon stem-loop that can mediate the stable coupling of the tRNA analogue to mRNA.

2. The tRNA analogue of claim 1, wherein the tRNA analogue is a 3′-amino-3′-deoxyadenosine-substituted tRNA or puromycin-substituted tRNA.

3. The tRNA analogue of claim 1, wherein the tRNA is yeast tRNA^{phe}.

4. The tRNA analogue of claim 3, wherein the 3′ terminal nucleotide of the yeast tRNA^{phe} has been replaced by 3′-amino-3′-deoxyadenosine or puromycin.

5. A tRNA analogue which is a tRNA in which the 3′ terminal nucleotide is replaced by 3′-amino-3′-deoxyadenosine and then linked to an amino acid moiety or replaced by puromycin and in which the anticodon loop comprises a reactive or activatible moiety that can mediate covalent coupling of the tRNA analogue to mRNA.

6. The tRNA analogue of claim 5, wherein the tRNA is yeast tRNA^{phe}.

7. The tRNA analogue of claim 5, wherein the reactive or activatible moiety is a modified base near or within the tRNA stem loop.

8. The tRNA analogue of claim 7 wherein the reactive or activatible moiety is a naturally modified guanine base at position 37.

9. A nonstandard polymer-tRNA analogue-mRNA fusion, comprising:
(a) a nonstandard polymer;
(b) a tRNA analogue comprising:
(i) a tRNA;
(ii) an amino acid moiety which can act as an acceptor substrate, but not as a donor substrate during translation;
(iii) a reactive or activatible moiety near or within the anticodon stem-loom that can mediate the stable coupling of the tRNA analogue to mRNA; and
(c) mRNA which encodes the nonstandard polymer of (a),
wherein the tRNA analogue is: located between the polymer and the mRNA; linked to the nonstandard polymer by a covalent bond between the terminal residue of the nonstandard polymer and the amino acid moiety; and linked to the mRNA by a covalent bond between a reactive or activatible moiety of the tRNA analogue and the mRNA.

10. The nonstandard polymer-tRNA analogue-mRNA fusion of claim 9, wherein the tRNA analogue is a 3′-amino-3′-deoxyadenosine-substituted tRNA or puromycin-substituted tRNA or a tRNA-bound analogue that acts as an acceptor but not a donor substrate during translation and the amino acid moiety comprises: an amino acid, and the methoxytyrosine moiety of puromycin.

11. The nonstandard polymer-tRNA analogue-mRNA fusion of claim 9, wherein the tRNA is yeast tRNA^{phe}.

12. The fusion of claim 11, wherein the yeast tRNA is yeast tRNA^{phe} in which the 3′ terminal nucleotide has been replaced by 3′-amino-3′-deoxyadenosine or puromycin.

13. A nonstandard polymer-tRNA analogue-mRNA fusion, comprising:
(a) a nonstandard polymer;
(b) a tRNA analogue comprising:
(i) a tRNA;
(ii) an amino acid moiety which can act as an acceptor substrate, but not as a donor substrate during translation; and
(iii) a reactive or activatible moiety near or within the anticodon stem-loop that can mediate the stable coupling of the tRNA analogue to mRNA; and
(c) mRNA which encodes the polymer of (a),
wherein the tRNA analogue is: located between the nonstandard polymer and the mRNA; linked to the nonstandard polymer by a covalent bond between the terminal residue of the nonstandard polymer and the amino acid moiety; and linked to the mRNA by the action of UV irradiation that produces a crosslink between a modified base near or within the tRNA stem loop and the mRNA.

14. The fusion of claim 13, wherein the tRNA analogue is a 3′-amino-3′-deoxyadenosine-substituted tRNA or puromycin-substituted tRNA or a tRNA-bound analogue that acts as an acceptor but not a donor substrate during translation and the amino acid moiety comprises: an amino acid, the methoxytyrosine moiety of puromycin-substituted tRNA.

15. The fusion of claim 13, wherein the tRNA is yeast tRNA^{phe}.

16. The fusion of claim 15, wherein the yeast tRNA is yeast tRNA^{phe} in which the 3′ terminal nucleotide has been replaced by 3′-amino-3′-deoxyadenosine or puromycin.

17. A nonstandard polymer-tRNA analogue-mRNA fusion, comprising:
(a) a nonstandard polymer;
(b) a tRNA analogue comprising:
(i) a tRNA;
(ii) an amino acid moiety which can act as an acceptor substrate, but not as a donor substrate during translation; and
(iii) a reactive or activatible moiety near or within the anticodon stem-loop that can mediate the stable coupling of the tRNA analogue to mRNA; and
(c) mRNA which encodes the polymer of (a),
wherein the tRNA analogue is: located between the nonstandard polymer and the mRNA; linked to the nonstandard polymer by a covalent bond between the terminal residue of the nonstandard polymer and the amino acid moiety; and linked to the mRNA by a covalent bond between the Y-base of the tRNA analogue and the mRNA.

18. The fusion of claim 17, wherein the tRNA analogue is a 3′-amino-3′-deoxyadenosine-substituted tRNA or puromycin-substituted tRNA or a tRNA-bound analogue that acts as an acceptor but not a donor substrate during translation and the amino acid moiety comprises: an amino acid, the methoxytyrosine moiety of puromycin-substituted tRNA.

19. The fusion of claim 17, wherein the tRNA is yeast tRNA^{phe}.

20. The fusion of claim 19, wherein the yeast tRNA is yeast tRNA^{phe} in which the 3′ terminal nucleotide has been replaced by 3′-amino-3′-deoxyadenosine or puromycin.

21. A diverse library of nonstandard polymers, wherein the nonstandard polymers comprise:
(a) a nonstandard polymer;
(b) a tRNA analogue comprising:
(i) a tRNA;
(ii) an amino acid moiety which can act as an acceptor substrate, but not as a donor substrate during translation; and
(iii) a reactive or activatible moiety near or within the anticodon stem-loop that can mediate the stable coupling of the tRNA analogue to mRNA; and
(c) mRNA which encodes the nonstandard polymer of (a),
wherein the tRNA analogue is: located between the nonstandard polymer and the mRNA; linked to the nonstandard polymer by a covalent bond between the terminal residue of the nonstandard polymer and the amino acid moiety; and linked to the mRNA by crosslinks between a reactive or activatible moiety of the tRNA analogue and the mRNA.

22. The diverse library of claim 21, wherein the tRNA analogue is a 3′-amino-3′-deoxyadenosine-substituted tRNA or puromycin-substituted tRNA or a tRNA-bound analogue that acts as an acceptor but not a donor substrate during translation and the amino acid moiety comprises: an amino acid, the methoxytyrosine moiety of puromycin.

23. The diverse library of claim 21, wherein the tRNA is yeast tRNA^{phe}.

24. The library of claim 23, wherein the 3′ terminal nucleotide of yeast tRNA^{phe} has been replaced by 3′-amino-3′-deoxyadenosine or puromycin.

25. A method of producing a diverse library of nonstandard polymers, which comprises nonstandard polymer-tRNA analogue-mRNA fusions, comprising the steps of:
(a) combining:
(i) mRNAs which encode nonstandard polymers;
(ii) tRNA analogues, wherein each tRNA analogue comprises:
(1) a tRNA;
(2) an amino acid moiety which can act as an acceptor substrate, but not as a donor substrate during translation; and
(3) a reactive or activatible moiety near or within the anticodon stem-loop that can mediate the stable coupling of the tRNA analogue to mRNA; and
(iii) an appropriate in vitro translation mixture, thereby producing a combination;
(b) maintaining the combination under conditions appropriate for translation of the mRNAs to produce the encoded nonstandard polymers and formation of a covalent amino acid-tRNA analogue bond between the terminal residue of a nonstandard polymer produced and the amino acid moiety present in the tRNA, to form nonstandard polymer-tRNA analogue fusions, thereby producing a mixture which contains stalled ribosomes that contain nonstandard polymer-tRNA analogue fusions; and
(c) exposing the mixture which contains stalled ribosomes that contain nonstandard polymer-tRNA analogue fusions to conditions which favor the crosslinking of the tRNA analogue and the mRNA which encodes the nonstandard polymer of the nonstandard polymer-tRNA analogue fusion,
whereby nonstandard polymer-tRNA analogue-mRNA fusions are produced, thereby producing a diverse library of nonstandard polymers.

26. The method of claim 25, wherein the tRNA analogue is a 3′-amino-3′-deoxyadenosine-substituted tRNA or puromycin-substituted tRNA and the amino acid moiety is any amino acid or the methoxytyrosine moiety of puromycin-substituted tRNA.

27. The method of claim 25, wherein the tRNA is yeast tRNA^{phe}.

28. The method of claim 27, wherein the 3′ terminal nucleotide of yeast tRNA^{phe} has been replaced with 3′-amino-3′-deoxyadenosine or puromycin and the conditions which favor crosslinking include mild ultraviolet irradiation.

29. A method of identifying members of a diverse library of encoded nonstandard polymers which exhibit a desired activity, wherein members are nonstandard polymer-tRNA analogue-mRNA fusions, comprising the steps of:
(a) producing a diverse library of encoded nonstandard polymers which comprises nonstandard polymer-tRNA analogue-mRNA fusions by:
(i) combining:
(1) mRNAs which encode nonstandard polymers;
(2) tRNA analogues, wherein each tRNA analogue comprises:
(a) a tRNA;
(b) an amino acid moiety which can act as an acceptor substrate, but not as a donor substrate during translation; and
(c) a reactive or activatible moiety near or within the anticodon stem-loop that can mediate the covalent coupling of the tRNA analogue to mRNA; and
(b) an appropriate in vitro translation mixture, thereby producing a combination;
(i) maintaining the combination under conditions appropriate for translation of the mRNAs to produce the encoded nonstandard polymers and formation of a covalent amino acid-tRNA analogue bond between the terminal residue of a nonstandard polymer produced and the amino acid moiety present in the tRNA analogue, to form nonstandard polymer-tRNA analogue fusions, thereby producing a mixture which contains stalled ribosomes that contain nonstandard polymer-tRNA analogue fusions; and
(ii) exposing the mixture which contains stalled ribosomes that contain nonstandard polymer-tRNA analogue fusions to conditions which favor the crosslinking the tRNA analogue and the mRNA which encodes the nonstandard polymer of the nonstandard polymer-tRNA analogue fusion, whereby nonstandard polymer-tRNA analogue-mRNA fusions are produced, thereby producing a diverse library of encoded nonstandard polymers;
(c) enriching the diverse library of encoded nonstandard polymers for members which exhibit a desired activity, thereby producing an enriched diverse library comprised of nonstandard polymer-tRNA analogue-mRNA fusions;
(d) amplifying the enriched diverse library by:
(i) reverse transcribing the mRNA components of the fusions, thereby producing the corresponding cDNA;
(ii) amplifying and transcribing in vitro the corresponding cDNA, thereby producing a pool of amplified, enriched mRNA from the corresponding cDNA;
(iii) combining the pool of amplified, enriched mRNA with an appropriate in vitro translation mixture and tRNA analogues of (a)(i)(2), thereby producing a combination;
(iv) maintaining the combination under conditions appropriate for translation of the mRNA to produce the encoded nonstandard polymers and formation of a covalent amino acid-tRNA analogue bond between the terminal residue of a nonstandard polymer produced and the amino acid moiety present in the tRNA analogue, to form nonstandard polymer-tRNA analogue fusions, thereby producing an amplified enriched mixture which contains stalled ribosomes that contain nonstandard polymer-tRNA analogue fusion; and
(v) exposing the amplified enriched mixture which contains stalled ribosomes that contain nonstandard polymer-tRNA analogue fusions to conditions which favor crosslinking of the tRNA analogue and the mRNA which encodes the nonstandard polymer of the nonstandard polymer-tRNA analogue fusion;
(e) repeating steps (b)-(c) as necessary until members which exhibit the desired activity are present in sufficient number to be detected; and
(f) detecting members which exhibit the desired activity, thereby identifying members which exhibit the desired activity.

30. The method of claim 29, wherein the tRNA analogue is a 3′-amino-3′-deoxyadenosine-substituted tRNA or puromycin-substituted tRNA or a tRNA-bound analogue that acts as an acceptor but not a donor substrate during translation and the amino acid moiety comprises: an amino acid, the methoxytyrosine moiety of puromycin.

31. The method of claim 29, wherein in the nonstandard polymer-tRNA analogue-mRNA fusion, the tRNA is yeast tRNA^{phe}.

32. The method of claim 31, wherein in the yeast tRNA^{phe}, the 3′ terminal nucleotide has been replaced by 3′-amino-3′-deoxyadenosine or puromycin.

33. A member of a diverse library of nonstandard polymers which exhibits a desired activity, identified by the method of claim 29.

34. A nonstandard polymer fragment of a member of a diverse library of nonstandard polymers, wherein the member exhibits a desired activity and is identified by the method of claim 29.

35. A tRNA analogue-mRNA fragment of a member of a diverse library of nonstandard polymers, wherein the member exhibits a desired activity and is identified by the method of claim 29.


SEQUENCE LISTING (15 sequences)

SEQ ID NO: 1 - DNA, 36 bp, E. Coli
taatacgactcactataggctacgtagctcagttgg

SEQ ID NO: 2 - DNA, 20 bp, E. Coli
tggtggctacgacgggattc

SEQ ID NO: 3 - DNA, 34 bp, plasmid PKsK12A
taatacgactcactataggtaaagtgtcattagc

SEQ ID NO: 4 - DNA, 22 bp, plasmid PKsK12A
ctttatcttcagaagaaaaacc

SEQ ID NO: 5 - protein, 10 aa, E. Coli
MKDYKDDDDK

SEQ ID NO: 6 - RNA, 69 nt, E. Coli
ggauccuaggaagcuugaaggagauauaccaugaaagacuacaaggacgacgacgacaaguauaaaguu

SEQ ID NO: 7 - RNA, 69 nt, E. Coli
ggauccuaggaagcuugaaggagauauaccaugaaagacuacaaggacgacgacgacaaguuuuuuuuu

SEQ ID NO: 8 - RNA, 69 nt, E. Coli
ggauccuaggaagcuugaaggagauauaccaugaaagacuacaaggacgacgacgacaaguuuaaaguu

SEQ ID NO: 9 - RNA, 69 nt, E. Coli
ggauccuaggaagcuugaaggagauauaccaugaaagacuacaaggacgacgacgacaaguauuuuguu

SEQ ID NO: 10 - RNA, 69 nt, E. Coli
ggauccuaggaagcuugaaggagauauaccaugaaagacuacaaggacgacgacgacaaguauaaauuu

SEQ ID NO: 11 - RNA, 55 nt, E. Coli
ggguuaacuuuagaaggagguaaaaaaaaugaaacgugaaaagacauuuuuuuuu

SEQ ID NO: 12 - RNA, 55 nt, E. Coli
ggguuaacuuuagaaggagguaaaaaaaaugaaacgugaaaagacagaacguaca

SEQ ID NO: 13 - RNA, 121 nt, E. Coli
ggguuaacuuuagaaggagguaaaaaaaaugaaacaccaucaccaccaucacggaaaucguuuuuucuuuuucuuuuuccgcuagcgucagggcuauucaccauuaacccacuagggcguu

SEQ ID NO: 14 - RNA, 112 nt, E. Coli
ggguuaacuuuagaaggagguaaaaaaaaugcguugcgaucacggaaaucguuuuuucuuuuucuuuuuccgcuagcgucagggcuauucaccauuaacccacuagggcguu

SEQ ID NO: 15 - RNA, 55 nt, E. Coli
ggguuaacuuuagaaggagguaaaaaaaauguuuaaagaaaaguuugaacguaca